(19) United States
(12) Patent Application Publication
(10) Pub. No.: US 2025/0107695 A1
(43) Pub. Date: Apr. 3, 2025

(54) OVERTUBE AND ENDOSCOPE SYSTEM

(71) Applicant: FUJIFILM Corporation, Tokyo (JP)

(72) Inventors: Takashi HARADA, Kanagawa (JP); Toshizumi TANAKA, Kanagawa (JP); Motohiko MATSUSHITA, Kanagawa (JP); Shozo IYAMA, Kanagawa (JP)

(73) Assignee: FUJIFILM Corporation, Tokyo (JP)

(21) Appl. No.: 18/890,770

(22) Filed: Sep. 20, 2024

(30) Foreign Application Priority Data
Sep. 29, 2023 (JP) 2023-170672

(51) Int. Cl. A61B 1/00; A61B 1/12

(52) U.S. Cl. CPC A61B 1/00135; A61B 1/00006; A61B 1/00057; A61B 1/00082; A61B 1/12

(57) ABSTRACT

In an overtube, an effective length of an outer tube is 1200 mm or longer and 1700 mm or shorter, an effective length of an inner tube is 1400 mm or longer and 2000 mm or shorter, a first maximum protrusion length of an endoscope insertion part from a distal end of the inner tube is 100 mm or longer and 550 mm or shorter, and a second maximum protrusion length of the inner tube from a distal end of the outer tube is 200 mm or longer and 700 mm or shorter. A total maximum protrusion length, which is a sum of the first and second maximum protrusion lengths, is 300 mm or longer and 800 mm or shorter, and a maximum protrusion length ratio, which is a ratio of the first maximum protrusion length to the second maximum protrusion length, is 0.15 or more and 1.0 or less.

### CROSS-REFERENCE TO RELATED APPLICATION

The present application claims priority under 35 U.S.C § 119(a) to Japanese Patent Application No. 2023-170672 filed on Sep. 29, 2023, which is hereby expressly incorporated by reference, in its entirety, into the present application.

### BACKGROUND OF THE INVENTION

### 1. Field of the Invention

The present disclosure relates to an overtube to be inserted into a body together with an insertion part of an endoscope and an endoscope system.

### 2. Description of the Related Art

In the related art, in the medical field, a technique of inserting an insertion part of an endoscope into an intestinal tract (large intestine or small intestine) and observing an inner wall surface of the intestinal tract, performing a diagnosis and a treatment, and the like has been performed. Since the intestinal tract is complicatedly bent, it is difficult to transmit a force to a distal end of the insertion part simply by pushing in the insertion part of the endoscope, and it is difficult to insert the endoscope into a deep portion.

Therefore, a so-called double balloon type endoscope device in which a balloon that can be expanded and contracted is provided in each of an insertion part of an endoscope and a distal end part of an overtube (also referred to as an insertion assisting tool) that covers the insertion part has been known (see, for example, WO2019/073886A). With the endoscope device, a balloon control device can individually control the expansion and contraction of each balloon by supplying and suctioning air into and from each balloon. As a result, the insertion part can be inserted into the deep portion of the complicatedly bent intestinal tract by alternately inserting the insertion part of the endoscope and the overtube into the intestinal tract while temporarily fixing each balloon individually at a predetermined timing.

### SUMMARY OF THE INVENTION

Meanwhile, in the double balloon type endoscope device, an insertion operation of alternately inserting the insertion part of the endoscope and the overtube, and a retraction operation of retracting the insertion part of the endoscope and the overtube toward a hand side are performed. In this case, it is necessary to share the operation such that an endoscopist operates the insertion part of the endoscope and an assistant operates the overtube. Therefore, there is a problem in that cooperation between the endoscopist and the assistant is required, which is a burden on a user, and usability is not necessarily good.

The present invention has been made in view of such circumstances, and an object of the present invention is to provide an overtube and an endoscope system that can reduce a burden on a user and improve usability.

The present disclosure includes the following inventions.

A first aspect relates to an overtube that is long and flexible, the overtube comprising: an inner tube having a first insertion path into which an endoscope insertion part is insertable; an outer tube having a second insertion path into which the inner tube is insertable; a first balloon provided at a distal end part of the inner tube; and a second balloon provided at a distal end part of the outer tube, in which an effective length of the outer tube is 1200 mm or longer and 1700 mm or shorter, an effective length of the inner tube is 1400 mm or longer and 2000 mm or shorter, in a case in which the endoscope insertion part is inserted into the first insertion path and the inner tube is inserted into the second insertion path, a total maximum protrusion length, which is a sum of a first maximum protrusion length of the endoscope insertion part from a distal end of the inner tube and a second maximum protrusion length of the inner tube from a distal end of the outer tube, is 300 mm or longer and 800 mm or shorter, the first maximum protrusion length is 100 mm or longer and 550 mm or shorter, the second maximum protrusion length is 200 mm or longer and 700 mm or shorter, and a maximum protrusion length ratio, which is a ratio of the first maximum protrusion length to the second maximum protrusion length, is 0.15 or more and 1.0 or less.

A second aspect relates to the overtube according to the first aspect, in which an axial length of at least one balloon of the first balloon or the second balloon is 50 mm or longer.

A third aspect relates to the overtube according to the second aspect, in which the balloon has a bulging part that is expandable and contractable, and an axial length of the bulging part is 30 mm or longer.

A fourth aspect relates to the overtube according to any one of the first to third aspects, further comprising: an inner tube gripping part provided at a base end part of the inner tube; and an outer tube gripping part provided at a base end part of the outer tube, in which the inner tube gripping part and the outer tube gripping part each have an axial length of 15 mm or longer and 200 mm or shorter.

A fifth aspect relates to the overtube according to any one of the first to fourth aspects, in which the inner tube has a stopper part for an inner tube in an inner tube protruding region that protrudes from the distal end of the outer tube, and the stopper part for an inner tube has a stopper surface for an inner tube that comes into contact with the distal end of the outer tube to restrict a movement of the inner tube toward an axial base end side with respect to the outer tube.

A sixth aspect relates to the overtube according to the fifth aspect, in which the stopper part for an inner tube is a large-diameter part having an outer diameter larger than an inner diameter of the second insertion path.

A seventh aspect relates to the overtube according to the fifth or sixth aspect, in which the stopper part for an inner tube is provided at a position that is on a base end side with respect to the first balloon and that is within 100 mm from the distal end of the inner tube toward the base end side.

An eighth aspect relates to the overtube according to any one of the fifth to seventh aspects, in which the stopper part for an inner tube is attachable to and detachable from the inner tube.

A ninth aspect relates to the overtube according to any one of the first to eighth aspects, in which the inner tube has a lubricant supply part that supplies a lubricant, and the lubricant supply part has an inner supply port that is open to an inner peripheral surface of the inner tube and an outer supply port that is open to an outer peripheral surface of the inner tube.

A tenth aspect relates to the overtube according to the ninth aspect, in which the inner tube has an injection port for the lubricant, and the injection port is in communication with the inner supply port and the outer supply port.

An eleventh aspect relates to the overtube according to any one of the first to tenth aspects, in which the inner tube has an indicator indicating a relative axial position to the outer tube.

A twelfth aspect relates to the overtube according to any one of the first to eleventh aspects, further comprising: an inner tube gripping part provided at a base end part of the inner tube; and an outer tube gripping part provided at a base end part of the outer tube.

A thirteenth aspect relates to the overtube according to the twelfth aspect, in which the inner tube gripping part has a first finger hook part on which an operator is able to hook a finger, and the outer tube gripping part has a second finger hook part on which the operator is able to hook a finger other than the finger.

A fourteenth aspect relates to the overtube according to the twelfth or thirteenth aspect, in which the inner tube gripping part is attachable to and detachable from the inner tube.

A fifteenth aspect relates to the overtube according to any one of the first to fourteenth aspects, further comprising: a relative movement detection unit that detects a relative axial movement between the outer tube and the inner tube.

A sixteenth aspect relates to the overtube according to the fifteenth aspect, in which the relative movement detection unit includes a proximity sensor or a position sensor.

A seventeenth aspect relates to the overtube according to any one of the first to sixteenth aspects, further comprising: a movement direction detection unit that detects a movement direction of an axial movement of the inner tube.

An eighteenth aspect relates to the overtube according to the seventeenth aspect, in which the movement direction detection unit includes an azimuth sensor or an angular velocity sensor.

A nineteenth aspect relates to the overtube according to any one of the first to eighteenth aspects, in which at least one tube of the inner tube or the outer tube includes a common pipe line having one end side in communication with a balloon provided in the tube out of the first balloon and the second balloon, an air supply pipe line and a suction pipe line that are branched at and connected to the other end side of the common pipe line, an air supply source pipe line in communication with an air supply source, a suction source pipe line in communication with a suction source, a pipe line switching part that selectively switches between an air supply state in which the air supply pipe line and the air supply source pipe line are in communication with each other and the suction pipe line and the suction source pipe line are blocked from each other, and a suction state in which the air supply pipe line and the air supply source pipe line are blocked from each other and the suction pipe line and the suction source pipe line are in communication with each other, a first communication path communicating between the air supply source pipe line and a space on an outer peripheral surface side or an inner peripheral surface side of the tube, and a second communication path communicating between the suction source pipe line and the space on the outer peripheral surface side or the inner peripheral surface side of the tube.

A twentieth aspect relates to an endoscope system comprising: the overtube according to any one of the first to nineteenth aspects; and a balloon control device that controls air supply and suction with respect to the first balloon and the second balloon, in which the overtube includes a relative movement detection unit that detects a relative axial movement between the outer tube and the inner tube, and a movement direction detection unit that detects a movement direction of an axial movement of the inner tube, the balloon control device includes at least one processor, and the processor is configured to start the suction with respect to the second balloon in a case in which the relative movement detection unit detects that the distal end of the outer tube and the distal end of the inner tube are moved in a spaced direction, start the air supply with respect to the second balloon in a case in which the relative movement detection unit detects that the distal end of the outer tube and the distal end of the inner tube are moved in an approaching direction, start the air supply with respect to the first balloon in a case in which the movement direction detection unit detects that the inner tube is moved toward an axial distal end side, and start the suction with respect to the first balloon in a case in which the movement direction detection unit detects that the inner tube is moved toward an axial base end side.

A twenty-first aspect relates to the endoscope system according to the twentieth aspect, further comprising: a remote controller connected to the balloon control device, in which notification of a detection result of each of the relative movement detection unit and the movement direction detection unit is sent to the balloon control device via the remote controller.

A twenty-second aspect relates to an endoscope system comprising: the overtube according to any one of the first to nineteenth aspects; and an endoscope having the endoscope insertion part, in which the endoscope insertion part has a stopper part for an endoscope insertion part in an endoscope insertion part protruding region that protrudes from the distal end of the inner tube, and the stopper part for an endoscope insertion part has a stopper surface for an endoscope insertion part that comes into contact with the distal end of the inner tube to restrict a movement of the endoscope insertion part toward an axial base end side with respect to the inner tube.

A twenty-third aspect relates to the endoscope system according to the twenty-second aspect, in which the stopper part for an endoscope insertion part is a large-diameter part having an outer diameter larger than an inner diameter of the first insertion path.

A twenty-fourth aspect relates to the endoscope system according to the twenty-second or twenty-third aspect, in which the stopper part for an endoscope insertion part is attachable to and detachable from the endoscope insertion part.

A twenty-fifth aspect relates to an endoscope system comprising: the overtube according to any one of the first to nineteenth aspects; an endoscope having the endoscope insertion part and an operating part installed consecutively on a base end side of the endoscope insertion part; and a holding member provided to be attachable to and detachable from the operating part and holding at least one tube of a first tube for performing air supply and suction with respect to the first balloon and a second tube for performing air supply and suction with respect to the second balloon.

A twenty-sixth aspect relates to an endoscope system comprising: the overtube according to any one of the first to nineteenth aspects; and an endoscope having the endoscope insertion part, in which an effective length of the endoscope insertion part is 2000 mm.

A twenty-seventh aspect relates to an endoscope system comprising: the overtube according to any one of the first to nineteenth aspects; and an endoscope having the endoscope insertion part, in which a third balloon is provided at a distal end part of the endoscope insertion part.

A twenty-eighth aspect relates to the endoscope system according to the twentieth or twenty-first aspect, further comprising: an endoscope having the endoscope insertion part, in which the endoscope insertion part has a stopper part for an endoscope insertion part in an endoscope insertion part protruding region that protrudes from the distal end of the inner tube, and the stopper part for an endoscope insertion part has a stopper surface for an endoscope insertion part that comes into contact with the distal end of the inner tube to restrict a movement of the endoscope insertion part toward an axial base end side with respect to the inner tube.

A twenty-ninth aspect relates to the endoscope system according to the twenty-eighth aspect, in which the stopper part for an endoscope insertion part is a large-diameter part having an outer diameter larger than an inner diameter of the first insertion path.

A thirtieth aspect relates to the endoscope system according to the twenty-eighth or twenty-ninth aspect, in which the stopper part for an endoscope insertion part is attachable to and detachable from the endoscope insertion part.

A thirty-first aspect relates to the endoscope system according to any one of the twentieth, twenty-first, or twenty-eighth to thirtieth aspects, further comprising: an endoscope having the endoscope insertion part and an operating part installed consecutively on a base end side of the endoscope insertion part; and a holding member provided to be attachable to and detachable from the operating part and holding at least one tube of a first tube for performing air supply and suction with respect to the first balloon and a second tube for performing air supply and suction with respect to the second balloon.

A thirty-second aspect relates to the endoscope system according to any one of the twentieth, twenty-first, or twenty-eighth to thirty-first aspects, further comprising: an endoscope having the endoscope insertion part, in which an effective length of the endoscope insertion part is 2000 mm.

A thirty-third aspect relates to the endoscope system according to any one of the twentieth, twenty-first, or twenty-eighth to thirty-second aspects, further comprising: an endoscope having the endoscope insertion part, in which a third balloon is provided at a distal end part of the endoscope insertion part.

A thirty-fourth aspect relates to an operation method of an overtube for operating the overtube according to any one of the first to nineteenth aspects, in which the overtube is operated in a state in which the overtube and the endoscope insertion part are inserted into an intestinal tract, the inner tube is inserted into the second insertion path such that a part of the inner tube is exposed from the distal end of the outer tube, and the endoscope insertion part is inserted into the first insertion path such that a part of the endoscope insertion part is exposed from the distal end of the inner tube, the operation method comprising: an inner tube pushing-in step of pushing in the inner tube such that the first balloon and the second balloon are spaced from each other in a range in which the distal end of the inner tube is located on a base end side with respect to the distal end of the endoscope insertion part in a state in which the first balloon is contracted; a first balloon expansion step of expanding the first balloon; an outer tube pushing-in step of pushing in the outer tube such that the second balloon approaches the first balloon in a state in which the second balloon is contracted; a second balloon expansion step of expanding the second balloon; and a retraction step of retracting the inner tube and the outer tube to pull in the intestinal tract in a state in which the first balloon and the second balloon are expanded and are close to each other.

A thirty-fifth aspect relates to the operation method of an overtube according to the thirty-fourth aspect, in which the second balloon expansion step and the retraction step are performed in parallel.

A thirty-sixth aspect relates to the operation method of an overtube according to the thirty-fourth or thirty-fifth aspect, in which the inner tube and the outer tube are operated by a second operator different from a first operator who operates the endoscope insertion part.

A thirty-seventh aspect relates to the operation method of an overtube according to any one of the thirty-fourth to thirty-sixth aspects, in which the inner tube has a stopper part for an inner tube in an inner tube protruding region that protrudes from the distal end of the outer tube, the stopper part for an inner tube has a stopper surface for an inner tube that comes into contact with the distal end of the outer tube to restrict a movement of the inner tube toward an axial base end side with respect to the outer tube, and the inner tube and the outer tube are retracted together by retracting the inner tube in a state in which the stopper surface for an inner tube is in contact with the distal end of the outer tube in the retraction step.

A thirty-eighth aspect relates to the operation method of an overtube according to the thirty-seventh aspect, in which the inner tube is operated by a second operator different from a first operator who operates the endoscope insertion part.

A thirty-ninth aspect relates to an insertion method of an endoscope insertion part including the operation method of an overtube according to any one of the thirty-fourth to thirty-eighth aspects, the insertion method comprising: an endoscope insertion part pushing-in step of pushing in the endoscope insertion part such that the distal end of the endoscope insertion part is spaced from the distal end of the inner tube in a state in which a part of the endoscope insertion part is exposed from the distal end of the inner tube.

A fortieth aspect relates to the insertion method of an endoscope insertion part according to the thirty-ninth aspect, further comprising: a repeat step of performing, in a case in which an operation including the inner tube pushing-in step, the first balloon expansion step, the outer tube pushing-in step, the second balloon expansion step, and the retraction step is defined as an overtube operation, the overtube operation at least one time, in which the endoscope insertion part pushing-in step is performed independently of the repeat step.

A forty-first aspect relates to the insertion method of an endoscope insertion part according to the fortieth aspect, in which the endoscope insertion part pushing-in step is performed in a case in which the pulled-in intestinal tract is accumulated on a base end side of the second balloon due to the repeat step.

A forty-second aspect relates to the insertion method of an endoscope insertion part according to the forty-first aspect, in which the endoscope insertion part pushing-in step is performed in a case in which the pulled-in intestinal tract is accumulated on the base end side of the second balloon due to the repeat step, and then the repeat step is further performed.

According to the aspects of the present invention, it is possible to reduce the burden on the user and to improve the usability.

### BRIEF DESCRIPTION OF THE DRAWINGS

FIG. 1 is a configuration diagram of an endoscope system according to a first embodiment.

FIG. 2 is an exploded view of an overtube.

FIG. 3 is a cross-sectional view of the overtube in a state in which an endoscope insertion part is inserted into the overtube.

FIG. 4 is an enlarged cross-sectional view showing a distal end part of the overtube.

FIG. 5 is a functional block diagram showing a balloon control device.

FIG. 6 is a flowchart showing an example of an insertion method of the endoscope insertion part using the overtube.

FIGS. 7A to 7D are explanatory diagrams showing examples of the insertion method of the endoscope insertion part using the overtube.

FIGS. 8A to 8C are explanatory diagrams showing examples of the insertion method of the endoscope insertion part using the overtube.

FIG. 9 is an explanatory diagram showing preferred dimensions of the overtube.

FIG. 10 is a table showing examples of dimensions of each part of the overtube.

FIG. 11 is an explanatory diagram showing a second embodiment.

FIG. 12 is an explanatory diagram showing a third embodiment.

FIG. 13 is a block diagram of a balloon control device according to a third embodiment.

FIG. 14 is a block diagram of another example of the balloon control device according the third embodiment.

FIG. 15 is an explanatory diagram showing a fourth embodiment.

FIG. 16 is an explanatory diagram showing a fifth embodiment.

FIG. 17 is an explanatory diagram showing a sixth embodiment.

FIG. 18 is an explanatory diagram showing the sixth embodiment.

FIG. 19 is an explanatory diagram showing a seventh embodiment.

FIG. 20 is an explanatory diagram showing an eighth embodiment.

FIG. 21 is an explanatory diagram showing the eighth embodiment.

FIG. 22 is an explanatory diagram showing a ninth embodiment.

### DESCRIPTION OF THE PREFERRED EMBODIMENTS

Hereinafter, embodiments of the present invention will be described with reference to the accompanying drawings.

### First Embodiment

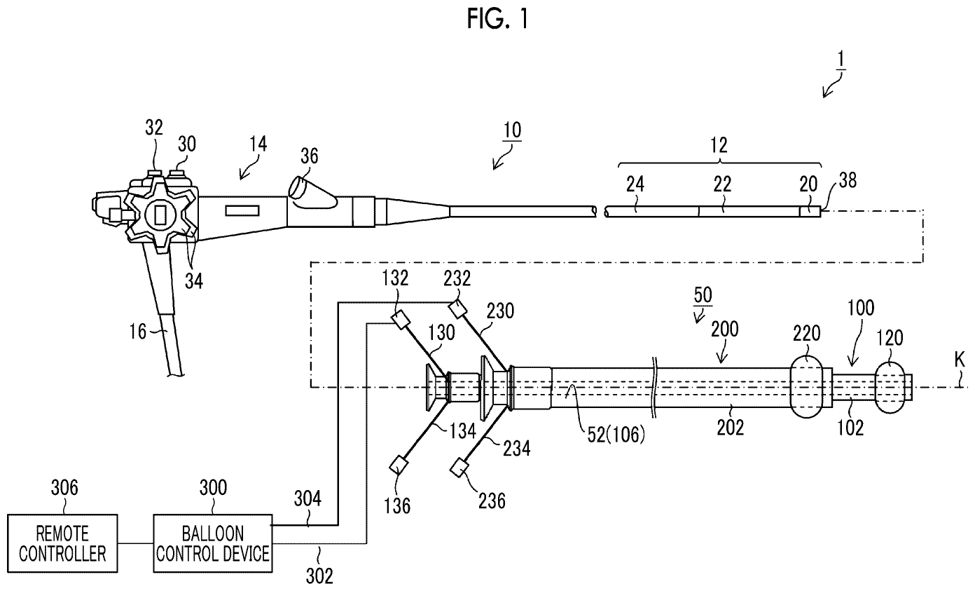

FIG. 1 is a configuration diagram of an endoscope system 1 according to a first embodiment. As shown in FIG. 1, the endoscope system 1 comprises an endoscope 10, an overtube 50, and a balloon control device 300.

### Endoscope

The endoscope 10 comprises an insertion part 12 (hereinafter, referred to as an “endoscope insertion part 12”) to be inserted into a body of a subject and an operating part 14 installed consecutively on a base end part of the endoscope insertion part 12.

One end of a universal cable 16 is connected to the operating part 14. Although not shown, a connector is provided at the other end of the universal cable 16, and the endoscope 10 and a light source device are connected to each other by using the connector. The light source device and a processor device are connected to each other, and the endoscope 10 and the processor device are connected to each other via the light source device.

The operating part 14 is provided with an air/water supply button 30, a suction button 32, and a pair of angle knobs 34 and 34. The operating part 14 is provided with a treatment tool inlet port 36 into which a treatment tool is inserted.

The endoscope insertion part 12 extends from a distal end of the operating part 14 and is formed to be thin and long as a whole. The endoscope insertion part 12 is composed of a distal end part 20, a bendable part 22, and a soft part 24 in this order from a distal end side to a base end side.

A distal end surface 38 of the distal end part 20 is provided with, although not shown, an observation window for observing the inside of the body, a pair of illumination windows for illuminating the inside of the body, an air/water supply nozzle for cleaning the observation window, and a forceps port for leading out the treatment tool.

An imaging element (not shown) is provided behind the observation window in the distal end part 20. An observation image is formed on the imaging element and is photoelectrically converted. A signal cable (not shown) is connected to the imaging element, and the signal cable is connected to the processor device via the endoscope insertion part 12, the operating part 14, the universal cable 16, and the like. Therefore, an electric signal indicating the observation image photoelectrically converted by the imaging element is output to the processor device, is appropriately subjected to signal processing, and is then output to a monitor. As a result, the observation image is displayed on the monitor.

In the distal end part 20, an emission end of a light guide (not shown) is disposed behind each of the pair of illumination windows. An incidence end of each light guide is connected to the light source device. As a result, illumination light supplied from the light source device to the incidence end of each light guide is applied to an observation site through the pair of illumination windows.

The bendable part 22 is bending-operated in a desired direction by operating the pair of angle knobs 34 and 34 provided in the operating part 14. A length of the bendable part 22 of the endoscope insertion part 12 in a longitudinal axis direction is, for example, 70 mm or longer and 150 mm or shorter. The soft part 24 occupies most of the endoscope insertion part 12 and is formed of a flexible member having flexibility. It should be noted that mm represents millimeters (the same applies hereinafter).

### Overtube

The overtube 50 is used in a case in which the endoscope insertion part 12 is inserted into the body of the subject. The overtube 50 is a member that is long and flexible. As will be described in detail below, the overtube 50 has a double structure in which two tubes (inner tube 100 and outer tube 200) are superimposed on each other, and the two tubes can be moved forward and backward with respect to each other so that an interval between balloons (first balloon 120 and second balloon 220) provided in the respective tubes can be changed.

As shown in FIG. 1, the overtube 50 is a long tubular member having flexibility, and is provided along a central axis K of the overtube 50. It should be noted that, in the following description, in a direction along the central axis K, one side (right side in FIG. 1) is defined as the distal end side, and the other side, which is a direction opposite to the one end side, is defined as the base end side.

An endoscope insertion path 52 into which the endoscope insertion part 12 can be inserted and which is formed along the central axis K is provided inside the overtube 50. The endoscope insertion path 52 is open to each end surface on the distal end side and the base end side in the central axis K direction, and the opening on the distal end side (distal end opening) and the opening on the base end side (base end opening) are in communication with each other. An inner diameter of the endoscope insertion path 52 is formed to be larger than an outer diameter of the endoscope insertion part 12. As a result, the endoscope insertion part 12 can be inserted into the endoscope insertion path 52 from the base end opening of the overtube 50, and a part of the endoscope insertion part 12 can be exposed from the distal end opening of the overtube 50.

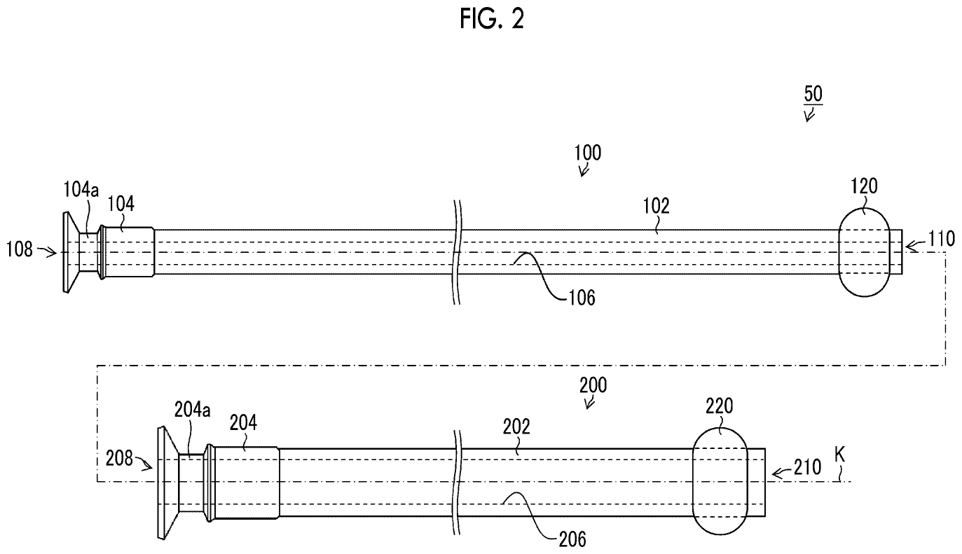

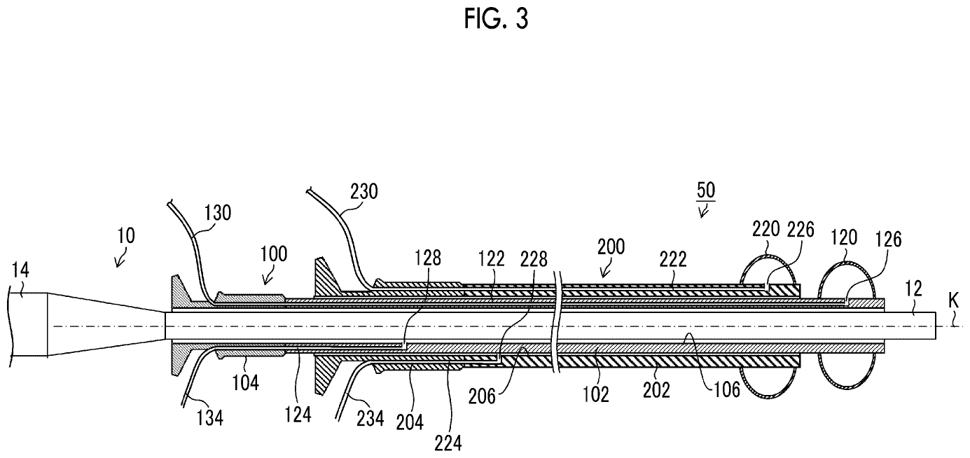

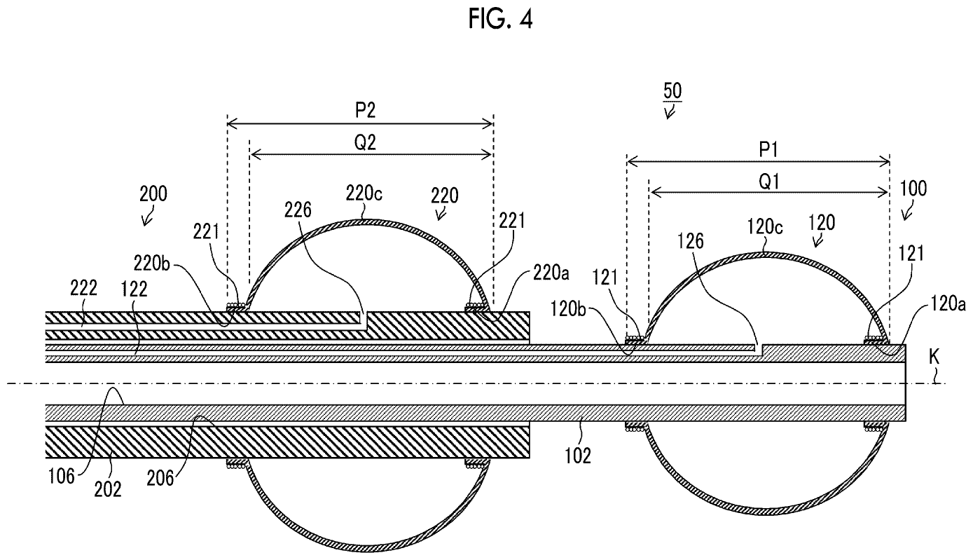

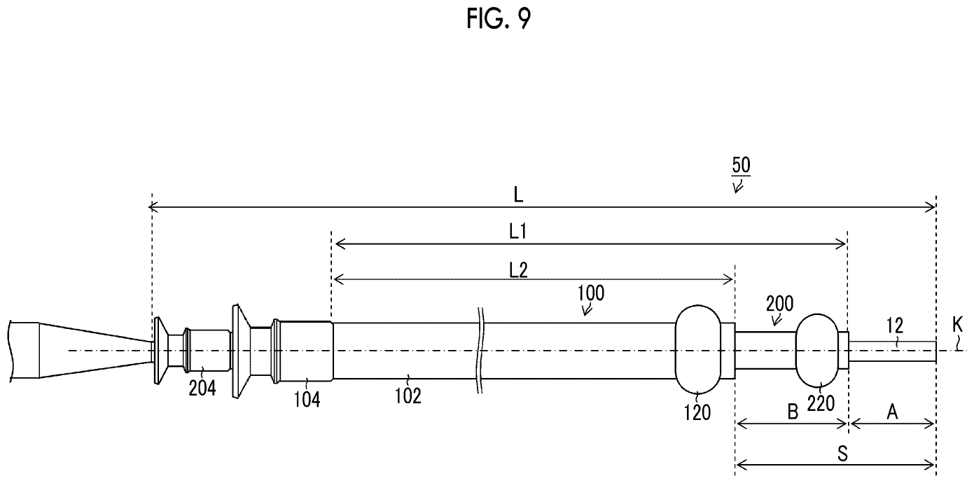

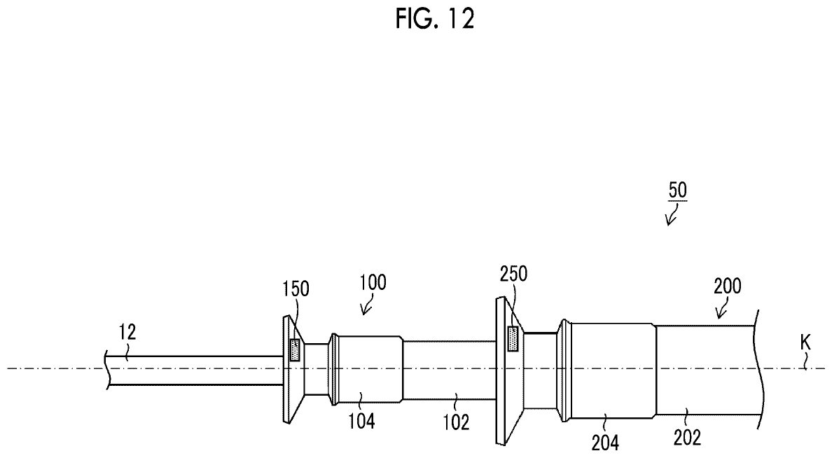

FIG. 2 is an exploded view of the overtube 50. FIG. 3 is a cross-sectional view of the overtube 50 in a state in which the endoscope insertion part 12 is inserted into the overtube 50. FIG. 4 is an enlarged cross-sectional view showing the distal end part of the overtube 50. It should be noted that, in FIG. 2, a first balloon air supply pipe 130, a second balloon air supply pipe 230, a first liquid supply pipe 134, and a second liquid supply pipe 234 are not shown for convenience. The same applies to other drawings (FIGS. 9, 12, and the like) described below.

As shown in FIGS. 2 to 4, the overtube 50 comprises the inner tube 100 and the outer tube 200. The overtube 50 is formed by the double structure in which the inner tube 100 and the outer tube 200 are superimposed on each other by inserting the inner tube 100 into an inside of the outer tube 200.

The inner tube 100 has an inner tube body 102. The inner tube body 102 is formed in a tubular shape by using various flexible materials and the like. An outer shape of the inner tube body 102 has a substantially constant size along the central axis K direction.

An inner tube gripping part 104 is attachably and detachably provided on the base end side of the inner tube body 102. The inner tube gripping part 104 is a part to be gripped by an operator (assistant) of the overtube 50, and is preferably formed to have a length (axial length) in the central axis K direction of 15 mm or longer and 200 mm or shorter. The inner tube gripping part 104 is formed in a tubular shape by using various hard materials. A fitting target part (not shown) is provided on the distal end side of the inner tube gripping part 104, and the inner tube body 102 is fitted and fixed to the fitting target part.

The inner tube gripping part 104 is provided with a small-diameter part 104a having a smaller outer diameter than the surroundings (see FIG. 2). The small-diameter part 104a is a part that is narrowed inward (toward the central axis K side) from the base end side and the distal end side in the central axis K direction in a side view, and the inner tube gripping part 104 is easily gripped by the operator by hooking a finger of the operator on the small-diameter part 104a. In addition, the small-diameter part 104a is likely to catch the finger of the operator, and also functions as an anti-slip part in a case in which the inner tube gripping part 104 is gripped.

A first insertion path 106 along the central axis K direction is provided inside the inner tube 100. The first insertion path 106 is provided from the inner tube gripping part 104 to the inner tube body 102. The first insertion path 106 is a pipe line (corresponding to the endoscope insertion path 52) into which the endoscope insertion part 12 can be inserted, and an inner diameter thereof is slightly larger than the outer diameter of the endoscope insertion part 12. A hydrophilic coating material (lubricating coating material) such as polyvinylpyrrolidone is applied on an inner peripheral surface of the first insertion path 106.

The inner tube 100 has a base end opening 108 provided at a base end of the inner tube 100 (base end of the inner tube gripping part 104) and a distal end opening 110 provided at a distal end of the inner tube 100 (distal end of the inner tube body 102), and the first insertion path 106 communicates between the base end opening 108 and the distal end opening 110. As a result, as shown in FIG. 3, the endoscope insertion part 12 is inserted into the first insertion path 106 from the base end opening 108 of the inner tube 100, and can be moved forward and backward along the central axis K direction, and a part of the region (region including at least the distal end part 20) of the endoscope insertion part 12 can be exposed from the distal end opening 110.

As shown in FIG. 4, the first balloon 120 formed of various elastic bodies is mounted on an outer peripheral surface of the inner tube body 102 on the distal end side. The first balloon 120 is mounted in a state of being penetrated by the inner tube body 102, and is composed of a bulging part 120c at a central part and attachment parts 120a and 120b at both ends thereof. The attachment part 120a on the distal end side is folded back inside the bulging part 120c, and a thread 121 is wound around the folded-back attachment part 120a. The attachment part 120b on the base end side is disposed on an outer side of the first balloon 120, and is fixed to the inner tube body 102 by winding the thread 121 therearound.

It is preferable that a length (axial length) P1 of the first balloon 120 in the central axis K direction is 50 mm or longer. It is preferable that a length (axial length) Q1 of the bulging part 120c in the central axis K direction is 30 mm or longer. In the first balloon 120 having such a length, in a case in which the bulging part 120c of the first balloon 120 is expanded, the first balloon 120 is locked to an inner wall surface of the intestinal tract without displacement, and thus the distal end part of the inner tube 100 can be stably fixed inside the intestinal tract.

In the first balloon 120 formed as described above, the bulging part 120c is expanded in a substantially spherical shape by blowing out air from a first air supply/suction port 126 described below, and the bulging part 120c is contracted by suctioning air from the first air supply/suction port 126.

A first air supply/discharge pipe line 122 and a first liquid pipe line 124 along the central axis K direction are formed on an outer peripheral wall part (wall part between the outer peripheral surface and the inner peripheral surface) of the inner tube 100 (inner tube body 102 and inner tube gripping part 104) (see FIGS. 3 and 4). The first air supply/discharge pipe line 122 is open as the first air supply/suction port 126 for the first balloon 120 on the outer peripheral surface located on an inner side of the first balloon 120. The first liquid pipe line 124 is open as the first liquid supply port 128 on the inner peripheral surface of the first insertion path 106 (inner peripheral surface of the inner tube body 102 or the inner tube gripping part 104).

The outer peripheral surface of the inner tube gripping part 104 is provided with the first balloon air supply pipe 130 in communication with the first air supply/discharge pipe line 122. As shown in FIG. 1, a first air supply port 132 is provided at one end of the first balloon air supply pipe 130. The first air supply port 132 is connected to the balloon control device 300 via a first connection tube 302. In a case in which the air is supplied from the balloon control device 300 into the first air supply port 132, the air is blown out from the first air supply/suction port 126 via the first balloon air supply pipe 130 and the first air supply/discharge pipe line 122. As a result, the first balloon 120 is expanded. In addition, in a case in which the air is suctioned by the balloon control device 300, the air in the first balloon 120 is suctioned from the first air supply/suction port 126 via the first air supply/discharge pipe line 122. As a result, the first balloon 120 is contracted.

The first liquid supply pipe 134 for supplying a lubricant such as water to the first insertion path 106 is provided on the outer peripheral surface of the inner tube gripping part 104. A first injection port 136 serving as an injection port for the lubricant is provided at one end of the first liquid supply pipe 134 (see FIG. 1). Lubricant supply means (not shown), such as a syringe, is connected to the first injection port 136. The lubricant supplied from the first liquid supply pipe 134 via the first injection port 136 is supplied from the first liquid supply port 128 to the inner peripheral surface of the first insertion path 106 via the first liquid pipe line 124, and reduces the friction with the outer peripheral surface of the endoscope insertion part 12 to be inserted into the first insertion path 106. It should be noted that the first liquid pipe line 124 and the first liquid supply port 128 constitute a lubricant supply part that supplies the lubricant.

The outer tube 200 has an outer tube body 202. The outer tube body 202 is formed in a tubular shape by using various flexible materials and the like. An outer shape of the outer tube body 202 has a substantially constant size along the central axis K direction.

An outer tube gripping part 204 is attachably and detachably provided on the base end side of the outer tube body 202. The outer tube gripping part 204 is a part to be gripped by the operator (assistant) of the overtube 50, and is preferably formed to have a length (axial length) in the central axis K direction of 15 mm or longer and 200 mm or shorter. The outer tube gripping part 204 is formed in a tubular shape by using various hard materials. A fitting target part (not shown) is provided on the distal end side of the outer tube gripping part 204, and the outer tube body 202 is fitted and fixed to the fitting target part.

The outer tube gripping part 204 is provided with a small-diameter part 204a having a smaller outer diameter than the surroundings (see FIG. 2). The small-diameter part 204a is a part that is narrowed inward (toward the central axis K side) from the base end side and the distal end side in the central axis K direction in a side view, and the outer tube gripping part 204 is easily gripped by the operator by hooking the finger of the operator on the small-diameter part 204a. In addition, the small-diameter part 204a is likely to catch the finger of the operator, and also functions as an anti-slip part in a case in which the outer tube gripping part 204 is gripped.

A second insertion path 206 along the central axis K direction is provided inside the outer tube 200. The second insertion path 206 is provided from the outer tube gripping part 204 to the outer tube body 202. The second insertion path 206 is a pipe line into which the inner tube body 102 of the inner tube 100 can be inserted, and an inner diameter thereof is slightly larger than an outer diameter of the inner tube body 102. A hydrophilic coating material (lubricating coating material) such as polyvinylpyrrolidone is applied on an inner peripheral surface of the second insertion path 206.

The outer tube 200 has a base end opening 208 provided at a base end of the outer tube 200 (base end of the outer tube gripping part 204) and a distal end opening 210 provided at a distal end of the outer tube 200 (distal end of the outer tube body 202), and the second insertion path 206 communicates between the base end opening 208 and the distal end opening 210. As a result, as shown in FIG. 3, the inner tube body 102 of the inner tube 100 is inserted into the second insertion path 206 from the base end opening 208 of the outer tube 200 and can be moved forward and backward along the central axis K direction, and a part of the region (region including at least the first balloon 120) of the inner tube 100 (inner tube body 102) can be exposed from the distal end opening 210.

As shown in FIG. 4, the second balloon 220 formed of various elastic bodies is mounted on an outer peripheral surface of the outer tube body 202 on the distal end side. The second balloon 220 is mounted in a state of being penetrated by the outer tube body 202, and is composed of a bulging part 220c at a central part and attachment parts 220a and 220b at both ends thereof. The attachment part 220a on the distal end side is folded back inside the bulging part 220c, and a thread 221 is wound around the folded-back attachment part 220a. The attachment part 220b on the base end side is disposed on an outer side of the second balloon 220, and is fixed to the outer tube body 202 by winding the thread 221 therearound.

It is preferable that a length (axial length) P2 of the second balloon 220 in the central axis K direction is 50 mm or longer. It is preferable that a length (axial length) Q2 of the bulging part 220c in the central axis K direction is 30 mm or longer. In the second balloon 220 having such a length, in a case in which the bulging part 220c of the second balloon 220 is expanded, the second balloon 220 is locked to the inner wall surface of the intestinal tract without displacement, and thus the distal end part of the outer tube 200 can be stably fixed inside the intestinal tract.

In the second balloon 220 formed as described above, the bulging part 220c is expanded in a substantially spherical shape by blowing out the air from a second air supply/suction port 226 described below, and the bulging part 220c is contracted by suctioning the air from the second air supply/suction port 226.

A second air supply/discharge pipe line 222 and a second liquid pipe line 224 along the central axis K direction are formed on an outer peripheral wall part (wall part between the outer peripheral surface and the inner peripheral surface) of the outer tube 200 (outer tube body 202 and outer tube gripping part 204) (see FIG. 3). The second air supply/discharge pipe line 222 is open as the second air supply/suction port 226 for the second balloon 220 on the outer peripheral surface located on an inner side of the second balloon 220. The second liquid pipe line 224 is open as the second liquid supply port 228 on the inner peripheral surface of the second insertion path 206 (inner peripheral surface of the outer tube body 202 or the outer tube gripping part 204).

The outer peripheral surface of the outer tube gripping part 204 is provided with the second balloon air supply pipe 230 in communication with the second air supply/discharge pipe line 222. As shown in FIG. 1, a second air supply port 232 is provided at one end of the second balloon air supply pipe 230. The second air supply port 232 is connected to the balloon control device 300 via a second connection tube 304. In a case in which the air is supplied from the balloon control device 300 into the second air supply port 232, the air is blown out from the second air supply/suction port 226 via the second balloon air supply pipe 230 and the second air supply/discharge pipe line 222. As a result, the second balloon 220 is expanded. In addition, in a case in which the air is suctioned by the balloon control device 300, the air in the second balloon 220 is suctioned from the second air supply/suction port 226 via the second air supply/discharge pipe line 222. As a result, the second balloon 220 is contracted.

The second liquid supply pipe 234 for supplying a lubricant such as water to the second insertion path 206 is provided on the outer peripheral surface of the outer tube gripping part 204. A second injection port 236 serving as an injection port for the lubricant is provided at one end of the second liquid supply pipe 234 (see FIG. 1). Lubricant supply means (not shown), such as a syringe, is connected to the second injection port 236. The lubricant supplied from the second liquid supply pipe 234 via the second injection port 236 is supplied from the second liquid supply port 228 to the inner peripheral surface of the second insertion path 206 via the second liquid pipe line 224, and reduces the friction with the outer peripheral surface of the inner tube 100 (inner tube body 102) to be inserted into the second insertion path 206.

As shown in FIG. 3, the overtube 50 is in a state in which the inner tube 100 and the outer tube 200 are integrally assembled together by inserting the inner tube body 102 of the inner tube 100 into the second insertion path 206 from the base end opening 208 of the outer tube 200 and exposing a part of the region (region including at least the first balloon 120) on the distal end side of the inner tube body 102 from the distal end opening 210 of the outer tube 200. In this case, the inner tube gripping part 104 is not inserted into the second insertion path 206 of the outer tube 200, and is located on the base end side (outside of the second insertion path 206) with respect to the base end opening 208 of the outer tube 200. As a result, it is possible to grip both the inner tube gripping part 104 and the outer tube gripping part 204, and the forward/backward movement operation of the inner tube 100 and the outer tube 200 can be performed by one operator.

Since the inner tube gripping part 104 is attachable to and detachable from the inner tube body 102, in a case of assembling the overtube 50, the inner tube body 102 may be inserted from the distal end opening 210 of the outer tube 200 into the second insertion path 206, a part of the region on the base end side of the inner tube body 102 may be exposed from the base end opening 208 of the outer tube 200, and then the inner tube body 102 may be fitted to the fitting target part of the inner tube gripping part 104. As a result, it is not necessary to insert the first balloon 120 into the second insertion path 206, and thus damage to the first balloon 120 can be prevented.

### Balloon Control Device

As shown in FIG. 1, the balloon control device 300 is connected to the first air supply port 132 of the inner tube 100 via the first connection tube 302 and is connected to the second air supply port 232 of the outer tube 200 via the second connection tube 304. A remote controller 306 is connected to the balloon control device 300. The balloon control device 300 supplies the air to each of the balloons 120 and 220 or suctions the air in each of the balloons 120 and 220 in response to a control signal from the remote controller 306. As a result, each of the balloons 120 and 220 is individually expanded or contracted.

A power switch, a stop switch, a pressure display unit for the first balloon 120, and a pressure display unit for the second balloon 220 are provided on a front panel of the balloon control device 300 (not shown). In addition, the first connection tube 302 that supplies and suctions the air to and from the first balloon 120 and the second connection tube 304 that supplies and suctions the air to and from the second balloon 220 are attached to the front panel of the balloon control device 300.

The remote controller 306 is connected to the balloon control device 300 by wire or wirelessly. The remote controller 306 is provided with a stop switch similar to the stop switch on the balloon control device 300 side, an ON/OFF switch for supporting the pressurization/decompression of the first balloon 120, a pause switch for holding a pressure of the first balloon 120, an ON/OFF switch for supporting the pressurization/decompression of the second balloon 220, and a pause switch for holding a pressure of the second balloon 220, and is configured to transmit and receive various signals including a signal corresponding to each switch to and from the balloon control device 300.

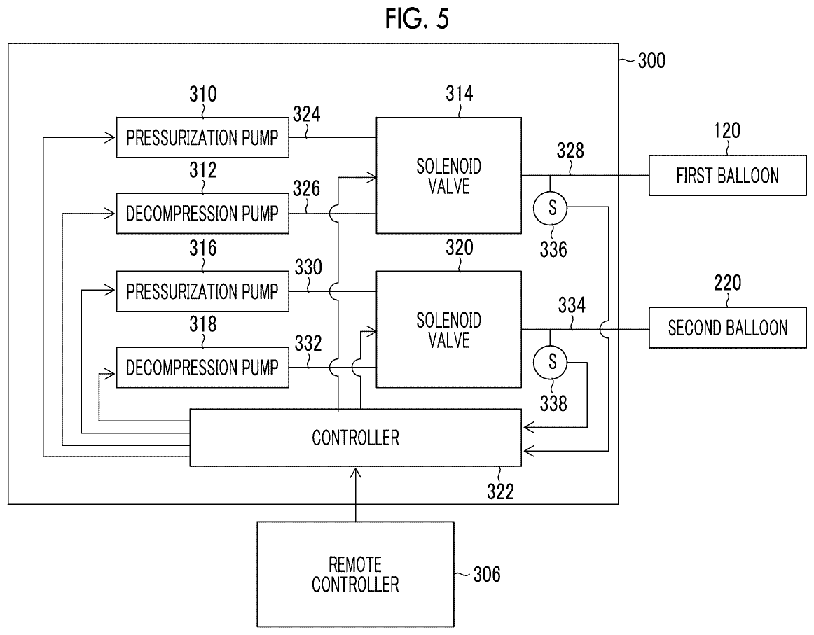

FIG. 5 is a functional block diagram showing the balloon control device 300. As shown in FIG. 5, the balloon control device 300 is provided with a pressurization pump 310, a decompression pump 312, a solenoid valve 314, and a pressure sensor 336, which are a control system of the first balloon 120; a pressurization pump 316, a decompression pump 318, a solenoid valve 320, and a pressure sensor 338, which are a control system of the second balloon 220; and a controller 322 that controls these pumps and solenoid valves.

The pressurization pump 310 and the decompression pump 312 are connected to the solenoid valve 314 via tubes 324 and 326, respectively. The solenoid valve 314 is in communication with the first balloon 120 via a tube 328. Therefore, by controlling the solenoid valve 314, one of the pressurization pump 310 or the decompression pump 312 can be put into communication with the first balloon 120. The pressure sensor 336 is connected to the tube 328, and the internal pressure of the first balloon 120 is measured by the pressure sensor 336. The measurement value of the pressure sensor 336 is displayed on the pressure display unit (not shown), and an electric signal indicating the measurement value is output to the controller 322.

Similarly, the pressurization pump 316 and the decompression pump 318 are connected to the solenoid valve 320 via tubes 330 and 332, respectively. The solenoid valve 320 is in communication with the second balloon 220 via a tube 334. Therefore, by controlling the solenoid valve 320, one of the pressurization pump 316 or the decompression pump 318 can be put into communication with the second balloon 220. The pressure sensor 338 is connected to the tube 334, and the internal pressure of the second balloon 220 is measured by the pressure sensor 338. The measurement value of the pressure sensor 338 is displayed on the pressure display unit (not shown), and an electric signal indicating the measurement value is output to the controller 322.

The controller 322 includes at least one processor described below, and functions as a controller that executes various types of control. The controller 322 controls the solenoid valve 314 based on the measurement value of the pressure sensor 336, to put one of the pressurization pump 310 or the decompression pump 312 into communication with the first balloon 120 and to drive the in-communication pressurization pump 310 or decompression pump 312. In addition, the controller 322 controls the solenoid valve 320 based on the measurement value of the pressure sensor 338, to put one of the pressurization pump 316 or the decompression pump 318 into communication with the second balloon 220 and to drive the in-communication pressurization pump 316 or decompression pump 318. As a result, the first balloon 120 and the second balloon 220 can be expanded by supplying the air or can be contracted by suctioning the air. In addition, by controlling the solenoid valves 314 and 320, both the pressurization pumps 310 and 316 and the decompression pumps 312 and 318 can be blocked from the first balloon 120 and the second balloon 220, so that the first balloon 120 and the second balloon 220 can be held in the expanded state or the contracted state.

### Insertion Method of Endoscope Insertion Part

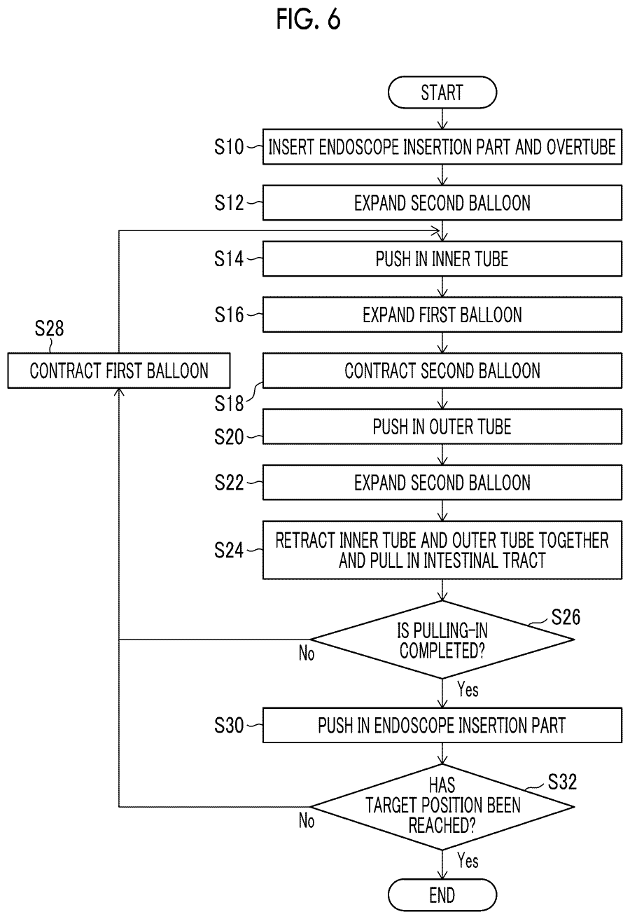

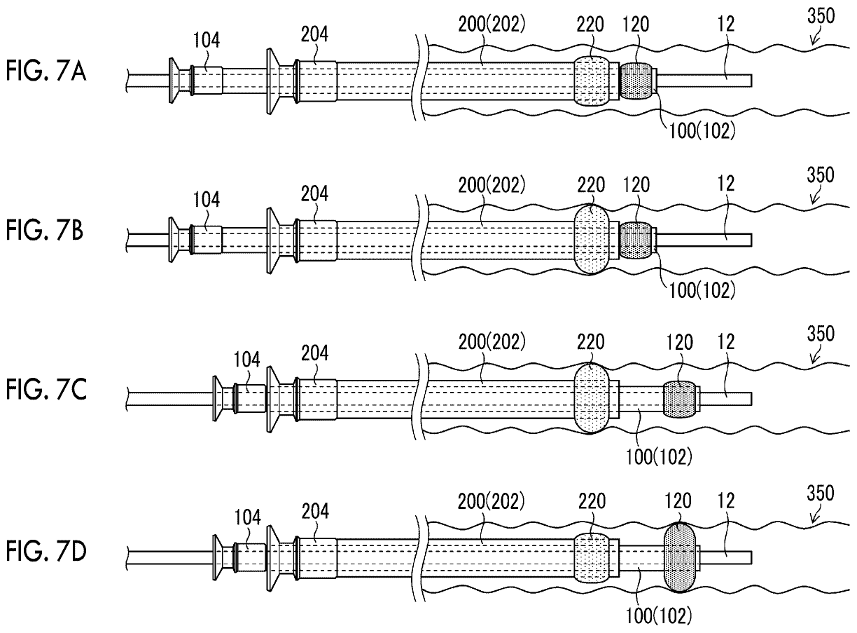

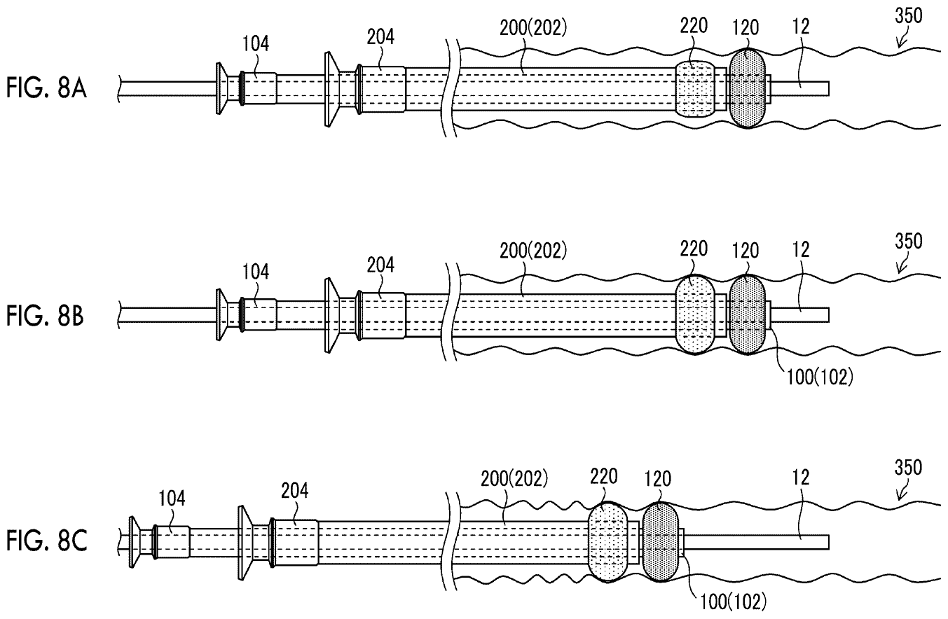

Next, an example of an insertion method of the endoscope insertion part 12 using the overtube 50 (including an operation method of the overtube) will be described. FIG. 6 is a flowchart showing an example of the insertion method of the endoscope insertion part 12 using the overtube 50. FIGS. 7 and 8 are explanatory diagrams showing examples of the insertion method of the endoscope insertion part 12 using the overtube 50. It should be noted that, in the following description, unless otherwise specified, the endoscope insertion part 12 is operated by a first operator (endoscopist), and the overtube 50 (inner tube 100 and outer tube 200) is operated by a second operator (assistant). In addition, the balloon control device 300 is operated by the second operator (or a third operator).

First, as shown in FIG. 7A, the endoscope insertion part 12 and the overtube 50 are inserted into an intestinal tract 350 from an anus or a mouth in a state in which the endoscope insertion part 12 is covered with the overtube 50 (that is, in a state in which the endoscope insertion part 12 is inserted into the first insertion path 106 of the inner tube 100) (step S10: initial insertion step). In this case, both the first balloon 120 and the second balloon 220 are in the contracted state. The distal end part 20 of the endoscope insertion part 12 is kept in a state of being exposed from the distal end opening 110 of the inner tube 100.

Next, as shown in FIG. 7B, the second balloon 220 is expanded by supplying the air from the balloon control device 300 to the second balloon 220 (step S12: second balloon expansion step). As a result, the second balloon 220 is locked to the inner wall surface of the intestinal tract 350, and the distal end part of the outer tube 200 (outer tube body 202) is fixed to the intestinal tract 350.

Next, as shown in FIG. 7C, the inner tube 100 is pushed in toward the deep portion of the intestinal tract 350 such that the first balloon 120 is spaced from the second balloon 220 while maintaining the states of the endoscope insertion part 12 and the outer tube 200, and is inserted along the endoscope insertion part 12 (step S14: inner tube pushing-in step). In this case, the inner tube 100 is pushed in a range in which the distal end of the inner tube 100 is located on the base end side with respect to the distal end of the endoscope insertion part 12.

Then, as shown in FIG. 7D, the first balloon 120 is expanded by supplying the air from the balloon control device 300 to the first balloon 120 (step S16: first balloon expansion step). As a result, the first balloon 120 is locked to the inner wall surface of the intestinal tract 350, and the distal end part of the inner tube 100 is fixed to the intestinal tract 350.

Next, as shown in FIG. 7D, the balloon control device 300 suctions the air from the second balloon 220 to contract the second balloon 220 (step S18: second balloon contraction step).

Thereafter, as shown in FIG. 8A, the outer tube 200 is pushed in such that the second balloon 220 approaches the first balloon 120 while maintaining the states of the endoscope insertion part 12 and the inner tube 100, and is inserted along the inner tube 100 (step S20: outer tube pushing-in step). In this case, the distal end of the outer tube 200 pushes in the inner tube 100 to a position at which the distal end of the outer tube 200 is not located beyond the base end of the first balloon 120 of the inner tube 100.

Then, after the distal end of the outer tube 200 is inserted into the vicinity of the first balloon 120, as shown in FIG. 8B, the second balloon 220 is expanded by supplying the air from the balloon control device 300 to the second balloon 220 (step S22: second balloon expansion step). As a result, the second balloon 220 is locked to the inner wall surface of the intestinal tract 350, and the distal end part of the outer tube 200 is fixed to the intestinal tract 350.

Next, as shown in FIG. 8C, the inner tube 100 and the outer tube 200 are retracted together toward the hand side in a state in which the first balloon 120 and the second balloon 220 are expanded and are close to each other, and the intestinal tract 350 is pulled in toward the hand side (step S24: pulling-in step).

Next, it is determined whether or not the pulling-in of the intestinal tract 350 is completed (step S26: pulling-in completion determination step). This determination is made by the second operator who operates the overtube 50 (may be made by the first operator who operates the endoscope insertion part 12). It should be noted that, in a case in which it is determined that the intestinal tract 350 pulled in toward the base end side of the second balloon 220 is accumulated, it is determined that the pulling-in of the intestinal tract 350 is completed. In a case in which it is determined that the pulling-in of the intestinal tract 350 is not completed (in a case of No in step S26), the first balloon 120 is contracted by suctioning the air from the first balloon 120 via the balloon control device 300 (step S28: first balloon contraction step), and then the processes from step S14 to step S26 are repeatedly performed. That is, an overtube operation including the inner tube pushing-in step, the first balloon expansion step, the second balloon contraction step, the outer tube pushing-in step, the second balloon expansion step, the pulling-in step, and the pulling-in completion determination step is repeatedly performed.

On the other hand, in a case in which it is determined that the pulling-in of the intestinal tract 350 is completed (in a case of Yes in step S26), the endoscope insertion part 12 is pushed-in toward the deep portion of the intestinal tract 350 in a state in which the first balloon 120 and the second balloon 220 are expanded (state shown in FIG. 8C) (step S30: endoscope insertion part pushing-in step). That is, in a state in which a part of the endoscope insertion part 12 is exposed from the distal end of the inner tube 100, the endoscope insertion part 12 is pushed in such that the distal end of the endoscope insertion part 12 is spaced from the distal end of the inner tube 100. As a result, the distal end part 20 of the endoscope insertion part 12 can be inserted into a deeper portion of the intestinal tract 350.

Next, it is determined whether or not the distal end of the endoscope insertion part 12 has reached a target position of the intestinal tract 350 (step S32: target position reached determination step). This determination is made by the first operator who operates the endoscope insertion part 12. In a case in which it is determined that the distal end of the endoscope insertion part 12 has not reached the target position of the intestinal tract 350 (in a case of No in step S32), the first balloon 120 is contracted by suctioning the air from the first balloon 120 via the balloon control device 300 (step S28: first balloon contraction step), and then the processes from steps S14 to S32 are repeatedly performed.

On the other hand, in a case in which it is determined that the distal end of the endoscope insertion part 12 has reached the target position of the intestinal tract 350 (in a case of Yes in step S32), the present flowchart ends.

In this way, by repeatedly performing the forward/backward movement operation of the inner tube 100 and the outer tube 200, it is possible to insert the endoscope insertion part 12 into the deep portion of the intestinal tract 350.

It should be noted that the operation of pushing in the endoscope insertion part 12 toward the deep portion of the intestinal tract 350 may be performed independently of the forward/backward movement operation (overtube operation) of the inner tube 100 and the outer tube 200. For example, in a case in which the forward/backward movement operation of the inner tube 100 and the outer tube 200 is repeatedly performed, the endoscope insertion part 12 may be pushed in toward the deep portion of the intestinal tract 350 in a state in which only the first balloon 120 out of the first balloon 120 and the second balloon 220 is expanded (state shown in FIG. 7D). In addition, in a state in which only the second balloon 220 out of the first balloon 120 and the second balloon 220 is expanded (state shown in FIG. 7B or 7C), the endoscope insertion part 12 may be pushed in toward the deep portion of the intestinal tract 350. However, since the endoscope insertion part 12 is inserted into the inner tube 100, it is preferable that the operation of pushing in the endoscope insertion part 12 toward the deep portion of the intestinal tract 350 is performed in a state in which at least the first balloon 120 is expanded and the distal end part of the inner tube 100 is fixed to the intestinal tract 350.

As described above, the overtube 50 according to the present embodiment is formed by the double structure in which the inner tube 100 and the outer tube 200 are superimposed on each other, and the first balloon 120 and the second balloon 220 are respectively provided at the distal end parts of the inner tube 100 and the outer tube 200, so that the operation required to pull in the intestinal tract can be performed by one operator.

In the overtube 50 according to the present embodiment, the forward/backward movement operation of the inner tube 100 and the outer tube 200 can be repeatedly performed with a short stroke, so that the intestinal tract can be efficiently pulled in toward the hand side.

In the overtube 50 according to the present embodiment, it is possible to perform the insertion operation of the endoscope insertion part 12 independently of the forward/backward movement operation of the inner tube 100 and the outer tube 200. Therefore, the operator (first operator) different from the operator (second operator) of the overtube 50 can operate the endoscope insertion part 12. Therefore, the burden in a case in which the operation of the endoscope insertion part 12 and the operation of the overtube 50 are performed in cooperation with each other can be significantly reduced, and the usability of the overtube 50 can be improved.

### Dimensions of Overtube

Next, preferred dimensions of the overtube 50 will be described with reference to FIG. 9. FIG. 9 is an explanatory diagram showing the preferred dimensions of the overtube 50.

It is preferable that the dimensions of the inner tube 100 and the outer tube 200 constituting the overtube 50 are determined based on a relationship between the ease of operation in a case in which one operator performs the forward/backward movement operation of the inner tube 100 and the outer tube 200 and a length of the intestinal tract into which the overtube 50 is inserted (maximum length from an insertion port (mouth or anus) of the subject to the deep portion of the intestinal tract) in the insertion method of the endoscope insertion part 12. It should be noted that, hereinafter, the description will be made on the premise that an effective length L of the endoscope insertion part 12 is 2000 mm.

As shown in FIG. 9, an effective length of the outer tube 200 is denoted by L2, and an effective length of the inner tube 100 is denoted by L1. In a case in which the endoscope insertion part 12 is inserted into the first insertion path 106 of the inner tube 100 and the inner tube 100 is inserted into the second insertion path 206 of the outer tube 200, a maximum protrusion length of the endoscope insertion part 12 from the distal end of the inner tube 100 (hereinafter, referred to as a “first maximum protrusion length”) is denoted by A, a maximum protrusion length of the inner tube 100 from the distal end of the outer tube 200 (hereinafter, referred to as a “second maximum protrusion length”) is denoted by B, and a total maximum protrusion length, which is the sum of the first maximum protrusion length A and the second maximum protrusion length B, is denoted by S (=A+B). In addition, a ratio of the first maximum protrusion length A to the second maximum protrusion length B, that is, a maximum protrusion length ratio, is R (=A/B).

It should be noted that the effective length L2 of the outer tube 200 is the maximum length of the outer tube 200 that can be inserted into the intestinal tract from the insertion port (mouth or anus) of the subject, and corresponds to a length of the outer tube body 202 in the central axis K direction excluding the outer tube gripping part 204 in the outer tube 200. The effective length L1 of the inner tube 100 is the maximum length of the inner tube 100 that can be inserted into the intestinal tract from the insertion port of the subject. That is, the effective length L1 of the inner tube 100 is a length of the inner tube body 102 in the central axis K direction in a case in which a base end position of the outer tube body 202 (distal end position of the outer tube gripping part 204), which is a reference of the length of the outer tube 200, is set as a reference position in a state in which the inner tube 100 is inserted into the outer tube 200 and the inner tube 100 is pushed in most toward the distal end side of the outer tube 200 (state in which the outer tube gripping part 204 and the inner tube gripping part 104 are closest to each other).

It is preferable that the dimensions of the respective parts of the overtube 50 according to the present embodiment satisfy the following conditions M1 to M6.

(M1) The effective length L2 of the outer tube 200 is 1300 mm or longer and 1600 mm or shorter.

(M2) The effective length L1 of the inner tube 100 is 1500 mm or longer and 1900 mm or shorter.

(M3) The total maximum protrusion length S is 400 mm or longer and 700 mm or shorter.

(M4) The first maximum protrusion length A is 100 mm or longer and 350 mm or shorter.

(M5) The second maximum protrusion length B is 200 mm or longer and 600 mm or shorter.

(M6) The maximum protrusion length ratio R is 0.15 or more and 1.0 or less.

These conditions M1 to M6 are derived in the following manner.

In the overtube 50 according to the present embodiment, in a case in which the forward/backward movement operation of the inner tube 100 and the outer tube 200 is performed, the total maximum protrusion length S is a reference for determining a stroke amount (forward/backward movement amount) of the inner tube 100 and the outer tube 200 required to pull in the intestinal tract, and is required to be generally 400 mm or longer and 700 mm or shorter. It should be noted that, in this range, 400 mm, which is the minimum value, is the minimum length required to pull in the intestinal tract. In addition, 700 mm, which is the maximum value, is obtained from a difference between the effective length L (2000 mm) of the endoscope insertion part 12 and the length (1300 mm) required for the outer tube 200 to exceed the length of the large intestine.

It is preferable that, in the condition (400 mm or longer and 700 mm or shorter) for the total maximum protrusion length S, the first maximum protrusion length A is equal to or shorter than the second maximum protrusion length B. In a case in which the first maximum protrusion length A is longer than the second maximum protrusion length B, the outer tube 200 is more easily moved in a case in which the inner tube 100 is pushed in than in a case of the opposite relationship. Therefore, it is desirable that the first maximum protrusion length A is the same as or shorter than the second maximum protrusion length B from the viewpoint of stably performing the forward/backward movement operation of the inner tube 100 and the outer tube 200.

### (1) Case in Which Total Maximum Protrusion Length S is Minimum Value

First, the effective length L1 of the inner tube 100 and the effective length L2 of the outer tube 200 required in a case in which the total maximum protrusion length S is 400 mm, which is the minimum value, will be considered. From the condition in which the first maximum protrusion length A is equal to or shorter than the second maximum protrusion length B, in a case in which the total maximum protrusion length S is 400 mm, for example, the second maximum protrusion lengths B in cases in which the first maximum protrusion lengths A are 100 mm and 200 mm are 300 mm and 200 mm, respectively. That is, the first maximum protrusion length A and the second maximum protrusion length B are determined such that the condition is satisfied in which the total maximum protrusion length S is 400 mm in a range in which the first maximum protrusion length A is 100 mm or longer and 200 mm or shorter and the second maximum protrusion length B is 200 mm or longer and 300 mm or shorter. It should be noted that, from the viewpoint of the operability of the endoscope insertion part 12 exposed from the distal end of the inner tube 100, the first maximum protrusion length A is required to be at least 100 mm or longer.

In a case in which the first maximum protrusion length A and the second maximum protrusion length B in a case in which the total maximum protrusion length S is 400 mm are determined as described above, the effective length L2 of the outer tube 200 is 1600 mm which is a value obtained by subtracting the total maximum protrusion length S from the effective length L (2000 mm) of the endoscope insertion part 12. In addition, the effective length L1 of the inner tube 100 is 1800 mm or longer and 1900 mm or shorter which is a value obtained by subtracting the first maximum protrusion length A (100 mm or longer and 200 mm or shorter) from the effective length L (2000 mm) of the endoscope insertion part 12. It should be noted that the effective length L1 of the inner tube 100 is equal to a value obtained by adding the second maximum protrusion length B to the effective length L2 (1600 mm) of the outer tube 200.

In a case in which the first maximum protrusion length A is 100 mm and the second maximum protrusion length B is 300 mm, the maximum protrusion length ratio R is 0.3 (=100/300). In a case in which the first maximum protrusion length A is 200 mm and the second maximum protrusion length B is 200 mm, the maximum protrusion length ratio R is 1.0 (=200/200). Therefore, the maximum protrusion length ratio R in a case in which the total maximum protrusion length S is 400 mm is a value in a range of 0.3 or more and 1.0 or less.

### (2) Case in Which Total Maximum Protrusion Length S is Maximum Value

Next, the effective length L1 of the inner tube 100 and the effective length L2 of the outer tube 200 required in a case in which the total maximum protrusion length S is 700 mm, which is the maximum value, will be considered. From the condition in which the first maximum protrusion length A is equal to or shorter than the second maximum protrusion length B, in a case in which the total maximum protrusion length S is 700 mm, for example, the second maximum protrusion lengths B in cases in which the first maximum protrusion lengths A are 100 mm, 200 mm, and 350 mm are 600 mm, 500 mm, and 350 mm, respectively. That is, the first maximum protrusion length A and the second maximum protrusion length B are determined such that the condition is satisfied in which the total maximum protrusion length S is 700 mm in a range in which the first maximum protrusion length A is 100 mm or longer and 350 mm or shorter and the second maximum protrusion length B is 350 mm or longer and 600 mm or shorter.

In a case in which the first maximum protrusion length A and the second maximum protrusion length B in a case in which the total maximum protrusion length S is 700 mm are determined as described above, the effective length L2 of the outer tube 200 is 1300 mm which is a value obtained by subtracting the total maximum protrusion length S from the effective length L (2000 mm) of the endoscope insertion part 12. In addition, the effective length L1 of the inner tube 100 is 1650 mm or longer and 1900 mm or shorter which is a value obtained by subtracting the first maximum protrusion length A (100 mm or longer and 350 mm or shorter) from the effective length L (2000 mm) of the endoscope insertion part 12. It should be noted that the effective length L1 of the inner tube 100 is equal to a value obtained by adding the second maximum protrusion length B to the effective length L2 (1300 mm) of the outer tube 200.

In a case in which the first maximum protrusion length A is 100 mm and the second maximum protrusion length B is 600 mm, the maximum protrusion length ratio R is 0.167 (=100/600). In a case in which the first maximum protrusion length A is 200 mm and the second maximum protrusion length B is 500 mm, the maximum protrusion length ratio R is 0.4 (=200/500). In a case in which the first maximum protrusion length A is 350 mm and the second maximum protrusion length B is 350 mm, the maximum protrusion length ratio R is 1.0 (=350/350). Therefore, the maximum protrusion length ratio R in a case in which the total maximum protrusion length S is 700 mm is a value in a range of 0.167 or more and 1.0 or less.

### (3) Case in Which Total Maximum Protrusion Length S is Minimum Value and Effective Length L2 of Outer Tube 200 is Minimum Value

In a case in which the total maximum protrusion length S is 400 mm, which is the minimum value, and the effective length L2 of the outer tube 200 is 1300 mm, which is the minimum value, in a case in which the first maximum protrusion length A is 200 mm and the second maximum protrusion length B is 200 mm, the effective length L1 of the inner tube 100 is the minimum value. In this case, the effective length L1 of the inner tube 100 is 1500 mm which is a value obtained by adding the second maximum protrusion length B (200 mm) to the effective length L2 of the outer tube 200.

In this way, the conditions M1 to M6 can be derived by selecting a range in which at least any of the ranges obtained in (1) to (3) described above is satisfied.

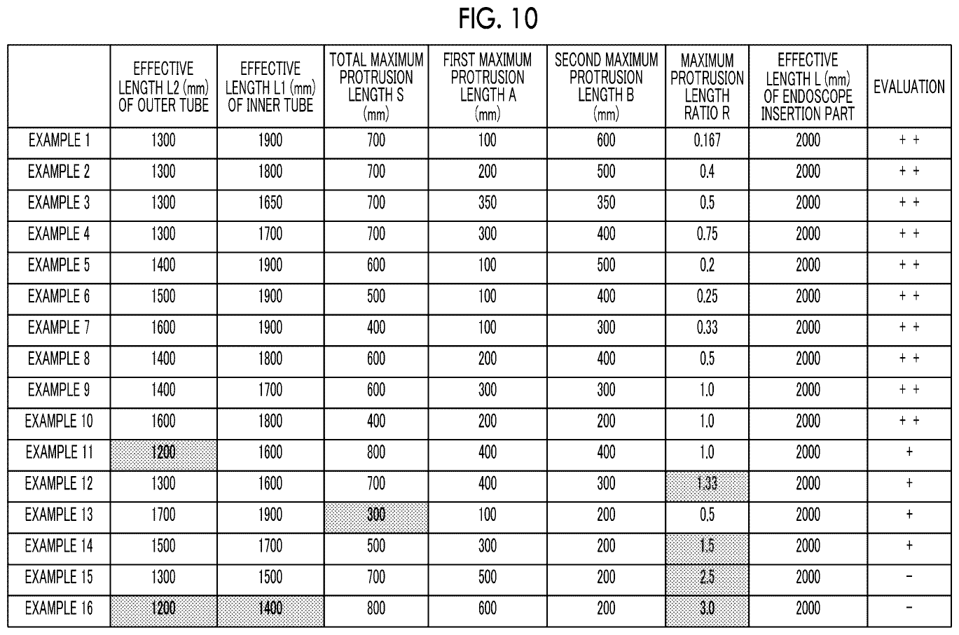

FIG. 10 is a table showing an example of the dimensions of each part of the overtube 50. It should be noted that, in FIG. 10, the numerical values that are deviated from the ranges defined by the conditions M1 to M6 are indicated by bold and shading. The evaluation in each example is made according to the following criteria.
++: All of the conditions are satisfied (excellent).
+: The value is slightly deviated from the ranges defined by some conditions (practicable).
−: The value is significantly deviated from the ranges defined by some conditions (impractical).

As shown in FIG. 10, Examples 1 to 10 satisfy all of the conditions M1 to M6. In this case, a length required to insert the overtube 50 (particularly, the outer tube 200) into the intestinal tract can be secured relative to the effective length L of the endoscope insertion part 12. In addition, it is possible to stably perform the forward/backward movement operation of the inner tube 100 and the outer tube 200 while securing the stroke amount (forward/backward movement amount) of the inner tube 100 and the outer tube 200 required to pull-in the intestinal tract.

Examples 11 and 14 are slightly deviated from the range defined by one condition among the conditions M1 to M6, and are slightly inferior to Examples 1 to 10, but are sufficient in practical use, and almost the same effect as in Examples 1 to 10 can be obtained.

Examples 15 and 16 indicate a case in which the maximum protrusion length ratio R exceeds 2.0 and is significantly deviated from the range defined by the condition M6. In such a case, as described above, the outer tube 200 is easily moved in a case in which the inner tube 100 is pushed in, which causes instability of the forward/backward movement operation of the inner tube 100 and the outer tube 200. It should be noted that, in Example 16, the effective length L2 of the outer tube 200 and the effective length L1 of the inner tube 100 are also slightly deviated from the ranges defined by the conditions M1 and M2, respectively.

In this way, since the dimensions of each part of the overtube 50 satisfy all of the conditions M1 to M6, the stroke amount required for the forward/backward movement operation between the inner tube 100 and the outer tube 200 can be obtained while securing the length required for the insertion of the overtube 50 into the intestinal tract. In addition, by determining the maximum protrusion length ratio R in the range defined by the condition M6, the first maximum protrusion length A is shorter than the second maximum protrusion length B, and the forward/backward movement operation of the inner tube 100 and the outer tube 200 can be stably performed.

It should be noted that the conditions M1 to M6 indicate preferred ranges in the present embodiment, but the values are not necessarily limited to the values in these ranges. For example, it is possible to obtain substantially the same effect as in the present embodiment even in the range indicated by the following conditions N1 to N6.

(N1) The effective length L2 of the outer tube 200 is 1200 mm or longer and 1700 mm or shorter.

(N2) The effective length L1 of the inner tube 100 is 1400 mm or longer and 2000 mm or shorter.

(N3) The total maximum protrusion length S is 300 mm or longer and 800 mm or shorter.

(N4) The first maximum protrusion length A is 100 mm or longer and 550 mm or shorter.

(N5) The second maximum protrusion length B is 200 mm or longer and 700 mm or shorter.

(N6) The maximum protrusion length ratio R is 0.15 or more and 1.0 or less.

The conditions M1 to M6 (as well as the conditions N1 to N6) have been described on the premise that the effective length L of the endoscope insertion part 12 is 2000 mm, but the present invention is not limited to this, and the conditions M1 to M6 (as well as the conditions N1 to N6) can also be applied to the endoscope insertion part 12 in which the effective length L is 1500 mm or longer and 2500 mm or shorter.

### Second Embodiment

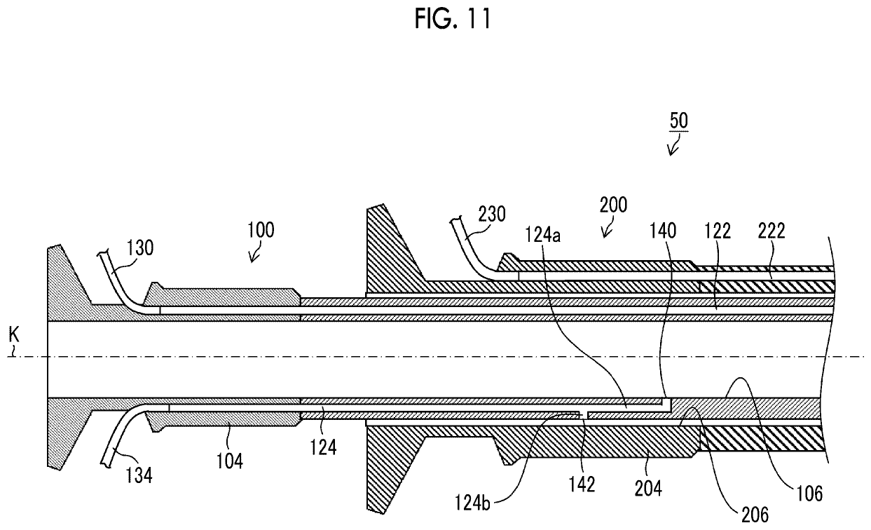

FIG. 11 is an explanatory diagram showing a second embodiment. The overtube 50 according to the second embodiment is the same as the overtube 50 according to the first embodiment except that the configuration of the lubricant supply part provided in the inner tube 100 is different. Hereinafter, the difference from the first embodiment will be described.

As shown in FIG. 11, in the second embodiment, the inner tube 100 comprises, as constituent elements of the lubricant supply part, an inner supply port 140 that is open to the inner peripheral surface (first insertion path 106) of the inner tube 100 and an outer supply port 142 that is open to the outer peripheral surface of the inner tube 100. It should be noted that a position of the outer supply port 142 in the central axis K direction is located on an inner side of the outer tube 200.

One end of the first liquid pipe line 124 is branched into two pipe lines in the outer peripheral wall part of the inner tube 100, and a branch pipe line 124a, which is one of the two branched pipe lines, is in communication with the inner supply port 140, and a branch pipe line 124b, which is the other thereof, is in communication with the outer supply port 142. The other end of the first liquid pipe line 124 is in communication with the first liquid supply pipe 134. As described above, the first liquid supply pipe 134 is provided with a first injection port 136 serving as an injection port for the lubricant at one end thereof (see FIG. 1). Therefore, the first injection port 136 is in communication with the inner supply port 140 and the outer supply port 142 via the first liquid supply pipe 134 and the first liquid pipe line 124.

According to the second embodiment, the lubricant supplied from the first liquid supply pipe 134 through the first injection port 136 can be supplied from the inner supply port 140 and the outer supply port 142 to both the inner peripheral surface of the first insertion path 106 (endoscope insertion path 52) of the inner tube 100 and the inner peripheral surface of the second insertion path 206 of the outer tube 200. Therefore, as shown in FIG. 11, it is not necessary to provide the pipe lines (the second liquid pipe line 224 and the second liquid supply pipe 234 according to the first embodiment, see FIG. 3) for supplying the lubricant in the outer tube 200 according to the second embodiment, and thus the structure of the outer tube 200 can be simplified.

### Third Embodiment

FIG. 12 is an explanatory diagram showing a third embodiment. The overtube 50 according to the third embodiment is the same as the overtube 50 according to the first embodiment except that a sensor is provided in each of the inner tube 100 and the outer tube 200. Hereinafter, the difference from the first embodiment will be described.

As shown in FIG. 12, the overtube 50 according to the third embodiment comprises a first sensor 150 provided in the inner tube 100 and a second sensor 250 provided in the outer tube 200.

The first sensor 150 functions as a movement direction detection unit that detects a movement direction of an axial movement (movement in the central axis K direction, and the same applies hereinafter) of the inner tube 100, and includes, for example, an azimuth sensor or an angular velocity sensor. The first sensor 150 is provided at any position in the inner tube 100. For example, the first sensor 150 may be provided in the inner tube gripping part 104 or in the inner tube body 102. It should be noted that it is desirable that the first sensor 150 is provided at a base end part of the inner tube gripping part 104, which is a position at which the movement direction of the axial movement of the inner tube 100 is easily detected, from the viewpoint of practicality.

The second sensor 250 functions as a relative movement detection unit that detects a relative axial movement between the outer tube 200 and the inner tube 100, and includes, for example, a proximity sensor or a position sensor. The second sensor 250 is provided at any position in the outer tube 200. For example, the second sensor 250 may be provided in the outer tube gripping part 204 or in the outer tube body 202. It should be noted that it is desirable that the second sensor 250 is provided at a base end part of the outer tube gripping part 204, which is a position at which the relative axial movement between the outer tube 200 and the inner tube 100 is easily detected. In addition, the second sensor 250 is not limited to being provided in the outer tube 200, and may be provided in the inner tube 100.

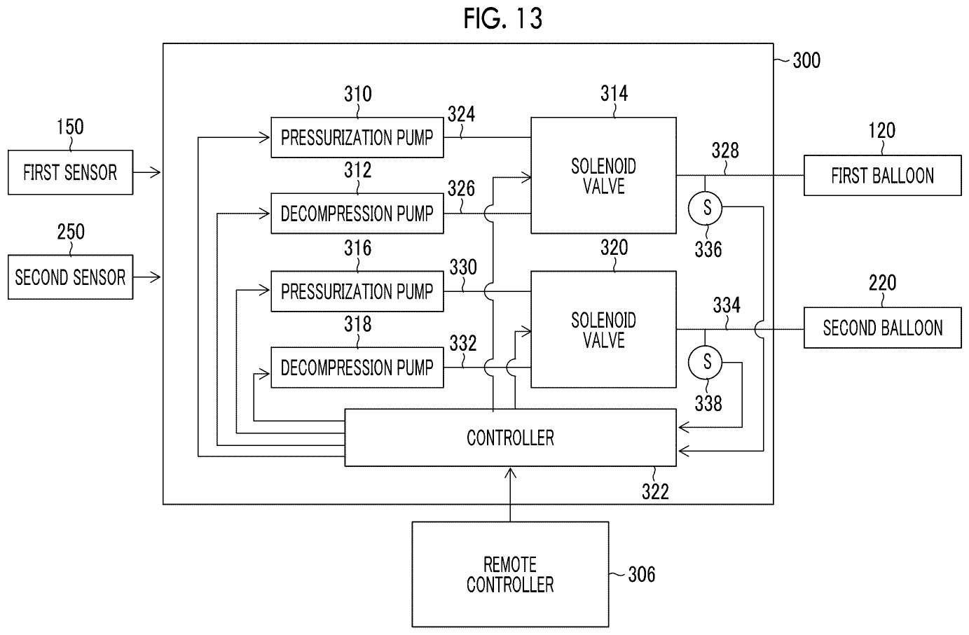

FIG. 13 is a block diagram of the balloon control device 300 according to the third embodiment. As shown in FIG. 13, the first sensor 150 and the second sensor 250 are connected to the balloon control device 300, and signals indicating detection results of these sensors are output to the controller 322.

The controller 322 controls the solenoid valve 314 based on the detection result of the first sensor 150. Specifically, in a case in which the first sensor 150 detects the movement of the inner tube 100 toward the axial distal end side, the controller 322 controls the solenoid valve 314 such that the pressurization pump 310 and the first balloon 120 are in communication with each other, and starts the air supply with respect to the first balloon 120. On the other hand, in a case in which the first sensor 150 detects the movement of the inner tube 100 toward the axial base end side, the controller 322 controls the solenoid valve 314 such that the decompression pump 312 and the first balloon 120 are in communication with each other, and starts the suction with respect to the first balloon 120.

The controller 322 controls the solenoid valve 320 based on the detection result of the second sensor 250. Specifically, in a case in which the second sensor 250 detects that the distal end of the outer tube 200 and the distal end of the inner tube 100 are moved in a spaced direction, the controller 322 controls the solenoid valve 320 such that the decompression pump 318 and the second balloon 220 are in communication with each other, and starts the suction with respect to the second balloon 220. On the other hand, in a case in which the second sensor 250 detects that the distal end of the outer tube 200 and the distal end of the inner tube 100 are moved in an approaching direction, the controller 322 controls the solenoid valve 320 such that the pressurization pump 316 and the second balloon 220 are in communication with each other, and starts the air supply with respect to the second balloon 220.

The insertion method of the endoscope insertion part 12 according to the third embodiment is essentially the same as the insertion method according to the first embodiment described above, but has the following different points.

As the first point, in the first embodiment, as shown in FIGS. 7B and 7C, the first balloon 120 needs to be expanded by operating the balloon control device 300 after pushing in the inner tube 100 in a state in which the first balloon 120 is contracted. In addition, as shown in FIGS. 8B and 8C, in a case in which the inner tube 100 is retracted together with the outer tube 200 and the forward/backward movement operation of the inner tube 100 and the outer tube 200 is repeatedly performed, it is necessary to operate the balloon control device 300 to contract the first balloon 120.

Meanwhile, in the third embodiment, since the first sensor 150 that functions as the movement direction detection unit that detects the movement direction of the axial movement of the inner tube 100 is provided, it is possible to detect the pushing-in of the inner tube 100 via the first sensor 150. Therefore, in a case in which the pushing-in of the inner tube 100 is started (see FIG. 7B), the first sensor 150 detects the movement of the inner tube 100 toward the axial distal end side, and the balloon control device 300 automatically starts the expansion of the first balloon 120. Then, the first balloon 120 is gradually expanded in parallel with the pushing-in of the inner tube 100, and the first balloon 120 is locked to the inner wall surface of the intestinal tract 350 in response to the completion of the pushing-in of the inner tube 100, so that the distal end part of the inner tube 100 is fixed to the intestinal tract 350 (see FIG. 7D).

In the third embodiment, since the first sensor 150 is provided, the retraction of the inner tube 100 can also be detected by the first sensor 150. Therefore, in a case in which the inner tube 100 starts to be retracted together with the outer tube 200 (see FIG. 8B), the first sensor 150 detects the movement of the inner tube 100 toward the axial base end side, and the contraction of the first balloon 120 is automatically started by the balloon control device 300. Then, the first balloon 120 is gradually contracted in parallel with the retraction of the inner tube 100, and the first balloon 120 is spaced from the inner wall surface of the intestinal tract 350 in response to the completion of the retraction of the inner tube 100, and thus the inner tube 100 can be immediately pushed in.

As the second point, in the first embodiment, as shown in FIGS. 7C, 7D, and 8A, in a case in which the outer tube 200 is pushed in, it is necessary to operate the balloon control device 300 to contract the second balloon 220 before the pushing-in is performed. In addition, as shown in FIGS. 8A to 8C, in a case in which the outer tube 200 is retracted together with the inner tube 100, it is necessary to operate the balloon control device 300 to expand the second balloon 220 before the retraction is performed.

Meanwhile, in the third embodiment, since the second sensor 250 that functions as the relative movement detection unit that detects the relative axial movement between the outer tube 200 and the inner tube 100 is provided, the pushing-in of the inner tube 100 can be detected by the second sensor 250. Therefore, in a case in which the pushing-in of the inner tube 100 is started (see FIGS. 7B and 7C), the second sensor 250 detects that the distal end of the inner tube 100 and the distal end of the outer tube 200 are moved in the spaced direction, and the contraction of the second balloon 220 is automatically started by the balloon control device 300. Then, the second balloon 220 is gradually contracted in parallel with the pushing-in of the inner tube 100, and the second balloon 220 is spaced from the inner wall surface of the intestinal tract 350 in response to the completion of the pushing-in of the inner tube 100, and thus the pushing-in operation of the outer tube 200 can be immediately performed (see FIG. 7D).

In the third embodiment, since the second sensor 250 is provided, the pushing-in of the outer tube 200 can be detected by the second sensor 250. Therefore, in a case in which the pushing-in of the outer tube 200 is started (see FIG. 7D), the second sensor 250 detects that the distal end of the inner tube 100 and the distal end of the outer tube 200 are moved in the approaching direction, and the expansion of the second balloon 220 is automatically started by the balloon control device 300. Then, the second balloon 220 is gradually expanded in parallel with the pushing-in of the outer tube 200, and the second balloon 220 is locked to the inner wall surface of the intestinal tract 350 in response to the completion of the pushing-in of the outer tube 200, so that the distal end part of the outer tube 200 is fixed to the intestinal tract 350 (see FIG. 8B).

As described above, according to the third embodiment, since the first sensor 150 and the second sensor 250 are provided, the expansion and contraction of the first balloon 120 and the second balloon 220 are automatically controlled by the balloon control device 300 in response to the forward/backward movement operation of the inner tube 100 and the outer tube 200. Therefore, it is not necessary to individually operate the expansion and contraction of the first balloon 120 and the second balloon 220, and the burden on the operator can be significantly reduced, and the operation efficiency of the overtube 50 can be improved.

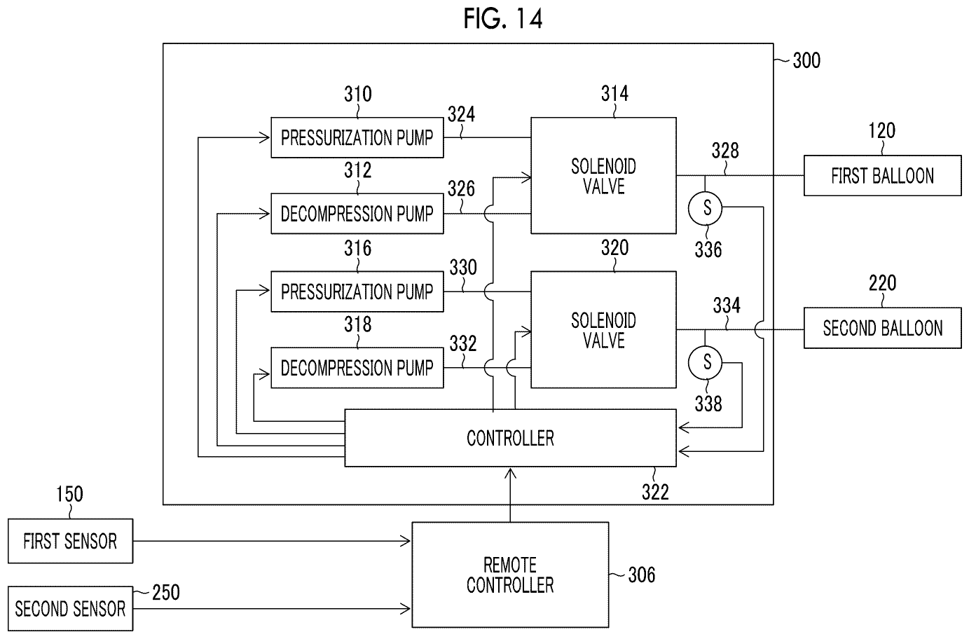

In addition, in the third embodiment, as an example, the configuration has been described in which the signals indicating the detection results of the first sensor 150 and the second sensor 250 are output to the balloon control device 300, but the present invention is not limited to this, and, for example, as shown in FIG. 14, the detection results of the first sensor 150 and the second sensor 250 may be output to the controller 322 of the balloon control device 300 via the remote controller 306. In this case, by disposing the remote controller 306 at a position close to the first sensor 150 and the second sensor 250, a wiring for connecting the first sensor 150 and the second sensor 250 to the balloon control device 300 is not required, and a degree of freedom in the disposition of the balloon control device 300 can be improved.

In addition, in the third embodiment, as an example, the case has been described in which the relative movement detection unit that detects the relative axial movement between the outer tube 200 and the inner tube 100 includes the second sensor 250 (proximity sensor or position sensor), but the present invention is not limited to this, and, for example, a magnet switch or an electric switch may be used.

### Fourth Embodiment

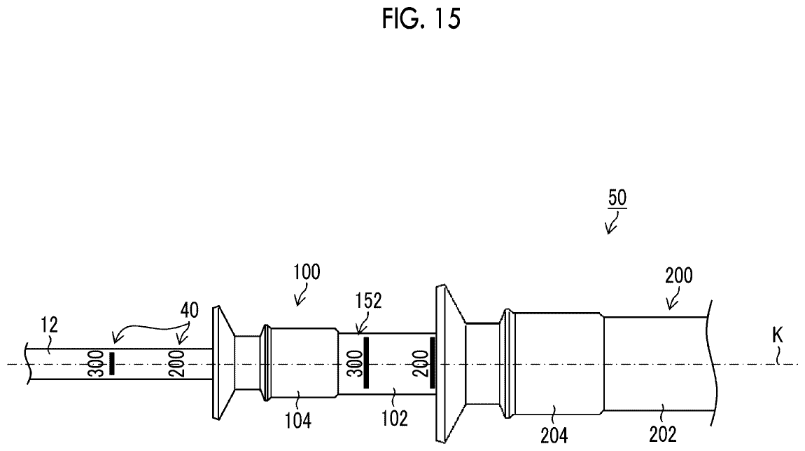

FIG. 15 is an explanatory diagram showing a fourth embodiment. The overtube 50 according to the fourth embodiment is the same as the overtube 50 according to the first embodiment except that an indicator is provided in each of the inner tube 100 and the endoscope insertion part 12. Hereinafter, the difference from the first embodiment will be described.

As shown in FIG. 15, the inner tube 100 is provided with an indicator 152 indicating a relative position to the outer tube 200 in the central axis K direction. The indicator 152 is provided on the outer peripheral surface of the inner tube body 102 and is composed of characters, figures, or a combination of characters and figures. The indicator 152 includes information related to the protrusion length of the inner tube 100 protruding from the distal end of the outer tube 200. In addition, the indicator 152 may include information indicating a position at which the inner tube 100 can be pushed in the outer tube 200 to the maximum extent.

The endoscope insertion part 12 is provided with an indicator 40 indicating a relative position to the inner tube 100 in the central axis K direction. The indicator 40 is provided on the outer peripheral surface of the endoscope insertion part 12 and is composed of characters, figures, or a combination of characters and figures. The indicator 40 includes information related to the protrusion length of the endoscope insertion part 12 protruding from the distal end of the inner tube 100.

According to the fourth embodiment, since the forward/backward movement operation of the inner tube 100 and the outer tube 200 is performed while checking the indicator 152 of the inner tube 100, the forward/backward movement operation can be performed while understanding the protrusion length of the inner tube 100 with respect to the outer tube 200, and thus the operation efficiency of the overtube 50 is improved.

In addition, according to the fourth embodiment, since the pushing-in operation of the endoscope insertion part 12 is performed while checking the indicator 40 of the endoscope insertion part 12, the pushing-in operation can be performed while understanding the protrusion length of the endoscope insertion part 12 with respect to the inner tube 100, and thus the operation efficiency of the endoscope insertion part 12 is improved.

It should be noted that, in the fourth embodiment, as an example, the case has been described in which the indicators 152 and 40 are respectively provided in the inner tube 100 and the endoscope insertion part 12, but the present invention is not limited to this, and the indicator may be provided in only one of the inner tube 100 or the endoscope insertion part 12.

### Fifth Embodiment

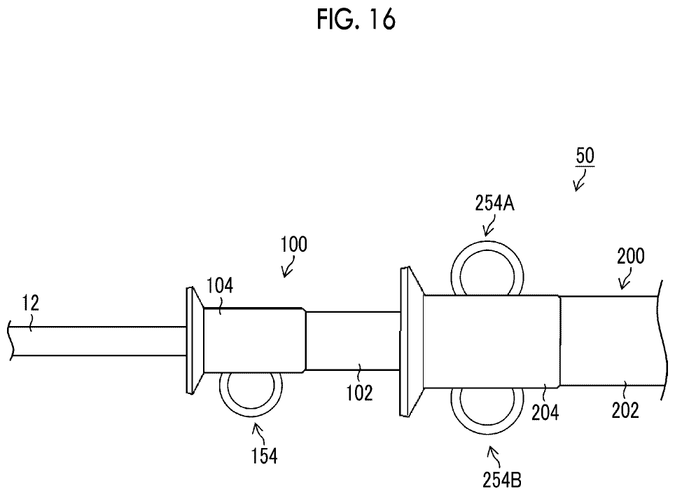

FIG. 16 is an explanatory diagram showing a fifth embodiment. The overtube 50 according to the fifth embodiment is the same as the overtube 50 according to the first embodiment except that the shapes of the inner tube gripping part 104 and the outer tube gripping part 204 are different. Hereinafter, the difference from the first embodiment will be described.

As shown in FIG. 16, the inner tube gripping part 104 and the outer tube gripping part 204 each have a cylindrical outer peripheral surface, and the small-diameter parts 104a and 204a as in the first embodiment are not provided.

In the fifth embodiment, a ring-shaped first finger hook part 154 on which a first finger (thumb) of the operator can be hooked is provided in the inner tube gripping part 104. In addition, the outer tube gripping part 204 is provided with ring-shaped second finger hook parts 254A and 254B on which a second finger (index finger) and a third finger (middle finger) of the operator can be hooked, respectively. The second finger hook part 254A and the second finger hook part 254B are disposed at positions facing each other by 180 degrees on the outer peripheral surface of the outer tube gripping part 204.

According to the fifth embodiment, the operator can easily perform the forward/backward movement operation of the inner tube 100 and the outer tube 200 with one hand by hooking the thumb on the first finger hook part 154 and hooking the index finger and the middle finger on the second finger hook parts 254A and 254B, respectively. As a result, the usability of the overtube 50 can be further improved.

### Sixth Embodiment

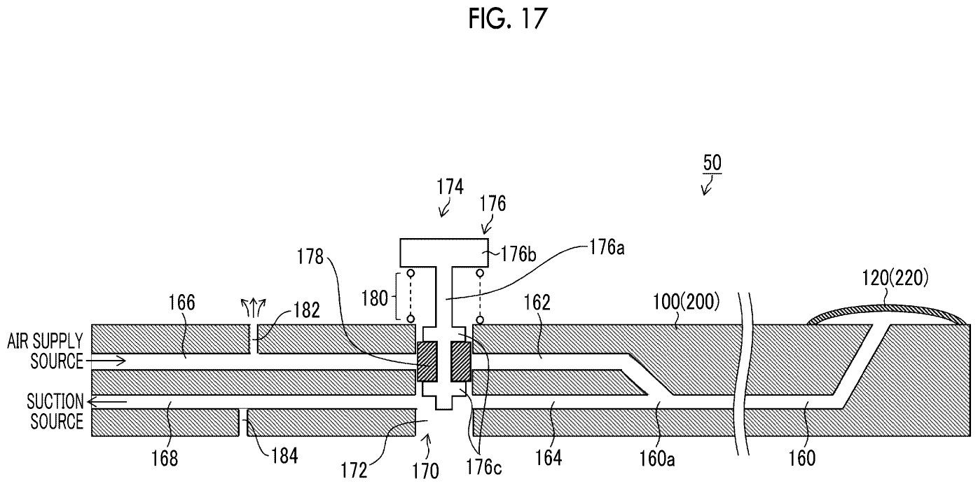

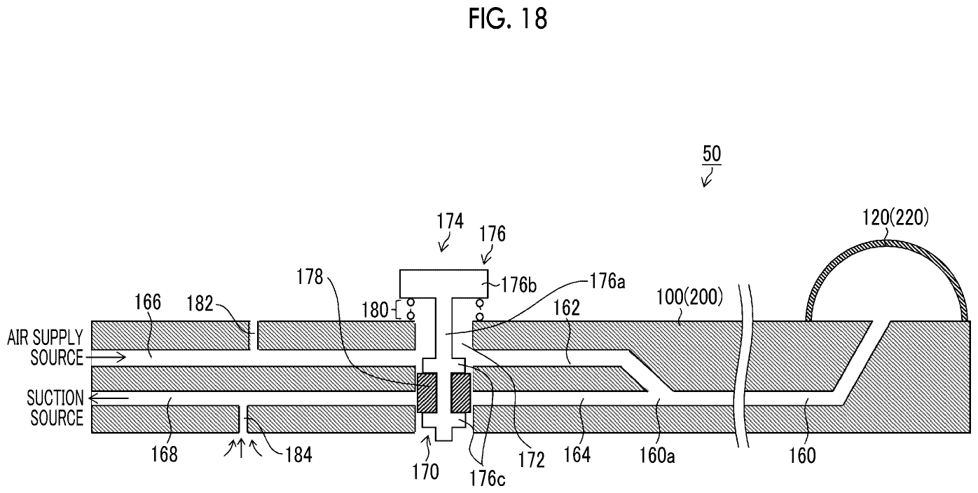

FIGS. 17 and 18 are explanatory diagrams showing a sixth embodiment. The overtube 50 according to the sixth embodiment is the same as the overtube 50 according to the first embodiment except that the overtube 50 according to the sixth embodiment has a configuration in which the pipe lines in communication with the first balloon 120 and the second balloon 220, respectively, can be selectively switched between an air supply state and a suction state. Hereinafter, the difference from the first embodiment will be described. It should be noted that, hereinafter, for the sake of description, a configuration will be described in which the first balloon 120 and the second balloon 220 can be selectively switched between the air supply state and the suction state by using the pipe line in communication with the first balloon 120 as a representative out of the first balloon 120 and the second balloon 220, but the same configuration also applies to the second balloon 220. In FIGS. 17 and 18, a part of the configuration of the inner tube 100 is shown in a simplified manner.

As shown in FIGS. 17 and 18, a common pipe line 160 having one end side in communication with the first balloon 120 is provided on the outer peripheral wall part of the inner tube 100 on which the first balloon 120 is mounted. The other end side of the common pipe line 160 is branched into two pipe lines at a branch part 160a. Among the two pipe lines, one pipe line is an air supply pipe line 162, and the other pipe line is a suction pipe line 164. That is, the air supply pipe line 162 and the suction pipe line 164 are branched at and connected to the other end side of the common pipe line 160.

An air supply source pipe line 166 in communication with an air supply source (pressurization pump) and a suction source pipe line 168 in communication with a suction source (decompression pump) are provided on the outer peripheral wall part of the inner tube 100. Air is always supplied from the air supply source to the air supply source pipe line 166. A first communication path 182 in communication with the outer peripheral surface of the inner tube 100 is provided in the middle of the air supply source pipe line 166. The first communication path 182 is a release hole that releases a part of a positive pressure generated in the suction source pipe line 168 in a case in which the air supply source pipe line 166 and the air supply pipe line 162 are in a non-communication state, and is formed to be smaller than a cross-sectional area of the air supply source pipe line 166. It should be noted that the first communication path 182 need only be in communication with any one of the outer peripheral surface or the inner peripheral surface of the inner tube 100.

Air is always suctioned by the suction source through the suction source pipe line 168. A second communication path 184 in communication with the inner peripheral surface of the inner tube 100 is provided in the middle of the suction source pipe line 168. The second communication path 184 is a release hole that releases a part of a negative pressure generated in the suction source pipe line 168 in a case in which the suction source pipe line 168 and the suction pipe line 164 are in a non-communication state, and is formed to be smaller than a cross-sectional area of the suction source pipe line 168. It should be noted that the second communication path 184 need only be in communication with any one of the outer peripheral surface or the inner peripheral surface of the inner tube 100.

The inner tube 100 is provided with a valve housing part 170. The valve housing part 170 is composed of a hole part that penetrates the outer peripheral wall part of the inner tube 100 from the outer peripheral surface to the inner peripheral surface. A communication space 172 is provided inside the valve housing part 170. The air supply pipe line 162, the suction pipe line 164, the air supply source pipe line 166, and the suction source pipe line 168 are in communication with the communication space 172 at predetermined positions.

A pipe line switching valve 174 is provided in the valve housing part 170. The pipe line switching valve 174 comprises a valve body part 176, a sealing member 178, and a spring member 180. The pipe line switching valve 174 is an example of a pipe line switching part.

The valve body part 176 is composed of a cylindrical shaft part 176a, a head part 176b provided at one end of the shaft part 176a, and a pair of flange parts 176c and 176c disposed on the other end side of the shaft part 176a with an interval therebetween. The sealing member 178 has a cylindrical shape, is provided between the pair of flange parts 176c and 176c, and is disposed coaxially with the shaft part 176a. The spring member 180 is interposed between the head part 176b and the outer peripheral surface of the inner tube 100. The spring member 180 biases the valve body part 176 toward the outside of the valve housing part 170 (that is, in a direction in which the head part 176b is spaced from the outer peripheral surface of the inner tube 100).

With such a configuration, in a state in which the valve body part 176 is not pressed by the operator (that is, in a non-operation state), the valve body part 176 is biased to a first position shown in FIG. 17 by a biasing force of the spring member 180. In this case, the sealing member 178 is located between the air supply pipe line 162 and the air supply source pipe line 166 in the communication space 172. Therefore, the air supply pipe line 162 and the air supply source pipe line 166 are in a non-communication state, and the suction pipe line 164 and the suction source pipe line 168 are in a communication state.

On the other hand, in a state in which the valve body part 176 is pressed by the operator (that is, in an operation state), the valve body part 176 is located at a second position shown in FIG. 18 against the biasing force of the spring member 180. In this case, the sealing member 178 is located between the suction pipe line 164 and the suction source pipe line 168 in the communication space 172. Therefore, the air supply pipe line 162 and the air supply source pipe line 166 are in a communication state, and the suction pipe line 164 and the suction source pipe line 168 are in a non-communication state.

Therefore, according to the sixth embodiment, the air supply state in which the air supply pipe line 162 and the air supply source pipe line 166 are in communication with each other and the suction pipe line 164 and the suction source pipe line 168 are blocked from each other, and the suction state in which the air supply pipe line 162 and the air supply source pipe line 166 are blocked from each other and the suction pipe line 164 and the suction source pipe line 168 are in communication with each other can be selectively switched by the presence or absence of the operation on the pipe line switching valve 174 (valve body part 176).

In addition, according to the sixth embodiment, since the first communication path 182 and the second communication path 184, which serve as release holes for preventing the pressure (positive pressure or negative pressure) from increasing, are provided in the middle of the air supply source pipe line 166 and the suction source pipe line 168, respectively, the air can be always supplied from the air supply source to the air supply source pipe line 166, and the air can be always suctioned by the suction source through the suction source pipe line 168. As a result, it is not necessary to provide the balloon control device 300 with the solenoid valve for selectively switching between the air supply source (pressurization pump) and the suction source (decompression pump), so that it is possible to simplify the structure of the balloon control device 300 and to reduce the cost.

It should be noted that, in the sixth embodiment, from the viewpoint of practicality, it is preferable that the pipe line switching valve 174 is disposed in the inner tube gripping part 104. However, the pipe line switching valve 174 is not limited to being disposed in the inner tube gripping part 104, and may be disposed in the inner tube body 102 as long as the pipe line switching valve 174 is disposed at a position at which the operator can perform the operation. The same applies to a case in which the pipe line switching valve 174 is provided in the outer tube 200.

### Seventh Embodiment

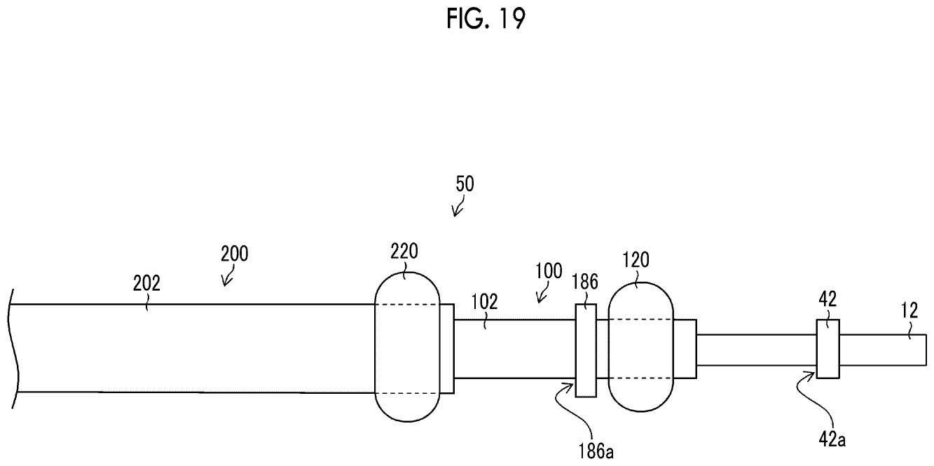

FIG. 19 is an explanatory diagram showing a seventh embodiment. The seventh embodiment is the same as the first embodiment except that the inner tube 100 and the endoscope insertion part 12 each comprise a stopper part for restricting the movement toward the axial base end side. Hereinafter, the difference from the first embodiment will be described.

As shown in FIG. 19, the inner tube 100 comprises a stopper part for an inner tube 186. The stopper part for an inner tube 186 is provided in an inner tube protruding region (region on the distal end side of the inner tube body 102) that protrudes from the distal end of the outer tube 200 (outer tube body 202). Specifically, the stopper part for an inner tube 186 is located at a position that is on the base end side with respect to the first balloon 120 and that is within 100 mm from the distal end of the inner tube 100 (inner tube body 102) toward the base end side.

The stopper part for an inner tube 186 is composed of a ring-shaped large-diameter part (for example, a rubber band) having an outer diameter larger than an inner diameter of the second insertion path 206 of the outer tube 200, and is attachable to and detachable from the inner tube 100 (inner tube body 102). The stopper part for an inner tube 186 has a stopper surface for an inner tube 186a on an end surface on a base end side thereof.

The stopper surface for an inner tube 186a comes into contact with the distal end (end surface on the distal end side) of the outer tube 200 to restrict the movement of the inner tube 100 toward the axial base end side with respect to the outer tube 200. From a different perspective, it can be said that the stopper surface for an inner tube 186a restricts the movement of the outer tube 200 toward the axial distal end side with respect to the inner tube 100.

In addition, as shown in FIG. 19, the endoscope insertion part 12 comprises a stopper part for an endoscope insertion part 42. The stopper part for an endoscope insertion part 42 is provided in an endoscope insertion part protruding region that protrudes from the distal end of the inner tube 100 (inner tube body 102).

The stopper part for an endoscope insertion part 42 is composed of a ring-shaped large-diameter part (for example, a rubber band) having an outer diameter larger than an inner diameter of the first insertion path 106 of the inner tube 100, and is attachable to and detachable from the endoscope insertion part 12. The stopper part for an endoscope insertion part 42 has a stopper surface for an endoscope insertion part 42a on an end surface on a base end side thereof.

The stopper surface for an endoscope insertion part 42a comes into contact with the distal end (end surface on the distal end side) of the inner tube 100 to restrict the movement of the endoscope insertion part 12 toward the axial base end side with respect to the inner tube 100. From a different perspective, it can be said that the stopper surface for an endoscope insertion part 42a restricts the movement of the inner tube 100 toward the axial distal end side with respect to the endoscope insertion part 12.

The insertion method of the endoscope insertion part 12 according to the seventh embodiment is essentially the same as the insertion method according to the first embodiment described above, but has the following different points.

In the first embodiment, as shown in FIGS. 8B and 8C, in a case in which the inner tube 100 and the outer tube 200 are retracted together, the operator (second operator) of the overtube 50 needs to retract the inner tube 100 and the outer tube 200 together in a state of gripping the inner tube gripping part 104 and the outer tube gripping part 204.

Meanwhile, in the seventh embodiment, since the stopper part for an inner tube 186 is provided in the inner tube 100, the outer tube 200 can also be retracted at the same time as the retraction of the inner tube 100 by retracting the inner tube 100 in a state in which the stopper surface for an inner tube 186a is in contact with the distal end of the outer tube 200. Therefore, the operator of the overtube 50 does not need to grip the outer tube gripping part 204, and thus it is possible to reduce the operation burden and to improve the operation efficiency.

According to the seventh embodiment, in a case in which the forward/backward movement operation of the inner tube 100 and the outer tube 200 is performed, the first balloon 120 of the inner tube 100 can be prevented from entering the inside of the outer tube 200 by the stopper part for an inner tube 186 provided in the inner tube 100.

In addition, according to the seventh embodiment, in a case in which the forward/backward movement operation of the endoscope insertion part 12 is performed independently of or in conjunction with the forward/backward movement operation of the inner tube 100 and the outer tube 200, it is possible to prevent the distal end part 20 of the endoscope insertion part 12 from entering the inside of the inner tube 100 via the stopper part for an endoscope insertion part 42 provided in the endoscope insertion part 12. As a result, it is possible to always expose the distal end part 20 of the endoscope insertion part 12 from the distal end of the inner tube 100 regardless of the forward/backward movement operation of the inner tube 100 and the outer tube 200, and thus the operability of the endoscope insertion part 12 is improved.

According to the seventh embodiment, the outer tube 200 can also be retracted at the same time as the retraction of the inner tube 100 by retracting the inner tube 100 in a state in which the inner tube gripping part 104 is gripped without gripping the outer tube gripping part 204. As a result, it is possible to reduce the operation burden and to improve the operation efficiency.

In the seventh embodiment, as an example, the case has been described in which the stopper part for an inner tube 186 and the stopper part for an endoscope insertion part 42 are each composed of a ring-shaped large-diameter part, but the present invention is not limited to this, and, for example, the stopper parts may have a C-shaped shape in which a part in a circumferential direction is cut out or a shape in which only a part in the circumferential direction protrudes in a radial direction.

In the seventh embodiment, the case has been described in which the stopper parts are provided in both the inner tube 100 and the endoscope insertion part 12, but the present invention is not limited to this, and the stopper part may be provided in only one of the inner tube 100 or the endoscope insertion part 12.

### Eighth Embodiment

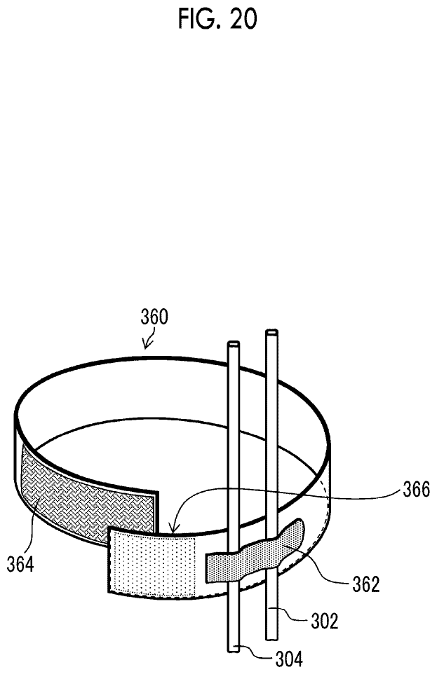

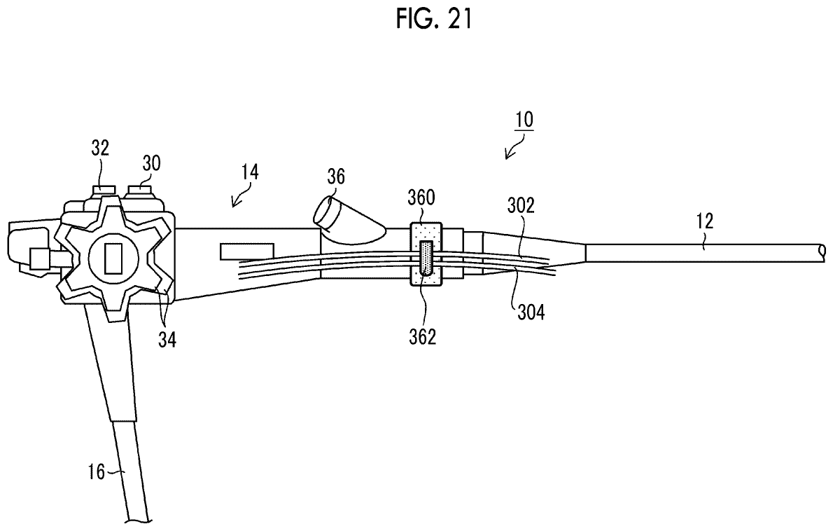

FIGS. 20 and 21 are explanatory diagrams showing an eighth embodiment. The eighth embodiment is the same as the first embodiment except that a holding member that holds the tube (first connection tube 302 and second connection tube 304) connecting the inner tube 100 and the outer tube 200 to the balloon control device 300 at the operating part 14 of the endoscope 10 is provided. Hereinafter, the difference from the first embodiment will be described.

As shown in FIG. 20, in the eighth embodiment, as an example of the holding member, a band member 360 having a band shape and flexibility is provided. The band member 360 has a length that can be wound around the operating part 14 of the endoscope 10. One end part of the band member 360 is attachable to and detachable from the other end part of the band member 360 by using a surface fastener. That is, a hook surface 364 of the surface fastener is provided on an outer side surface of one end part of the band member 360, and a loop surface 366 of the surface fastener is provided on an inner side surface of the other end part thereof, and these surfaces come into contact with each other to be attachably and detachably bonded to each other. It should be noted that the hook surface 364 and the loop surface 366 may be reversed. In addition to the surface fastener, various known configurations can be applied as long as the end parts of the band member 360 can be attachably and detachably bonded to each other. For example, a protrusion-like engaging member may be provided on one of an inner side surface of one end part and an outer side surface of the other end part of the band member 360, one or a plurality of engaging holes may be provided on the other thereof, and the engaging member may be engaged with the engaging hole to attachably and detachably bond the inner side surface and the outer side surface to each other.

A tube holding part 362 is provided on an outer side surface of the band member 360. The tube holding part 362 is composed of an elongated member having a narrower width than the band member 360. One end part of the band member 360 is fixed to the band member 360, and the other end part thereof is attachable to and detachable from the outer side surface of the band member 360 by using a surface fastener. The configuration of the surface fastener is the same as the configuration of the band member 360, and thus the description thereof will be omitted. It should be noted that various known configurations can be applied as long as the other end part of the band member 360 can be attachably and detachably bonded to the outer side surface of the band member 360.

In a case in which the first connection tube 302 and the second connection tube 304 are held by the tube holding part 362, the other end part of the tube holding part 362 is removed from the outer side surface of the band member 360. The first connection tube 302 and the second connection tube 304 are disposed between the tube holding part 362 and the outer side surface of the band member 360, and the other end part of the tube holding part 362 and the outer side surface of the band member 360 are brought into contact with each other to be bonded to each other, whereby the other end part of the tube holding part 362 is in a state of being closed. As a result, the first connection tube 302 and the second connection tube 304 are held between the tube holding part 362 and the outer side surface of the band member 360. It should be noted that the first connection tube 302 and the second connection tube 304 are examples of a first tube and a second tube according to the embodiment of the present invention, respectively.

With the band member 360 formed as described above, as shown in FIG. 21, the band member 360 can be wound around the outer peripheral portion of the operating part 14 of the endoscope 10 to bond the end parts of the band member 360 to each other by using the surface fastener, so that the band member 360 can be attachably and detachably mounted on the operating part 14 while the length of the band member 360 is adjusted. The first connection tube 302 and the second connection tube 304 are held by the tube holding part 362. As a result, it is possible to dispose the first connection tube 302 and the second connection tube 304 at a position along the operating part 14 of the endoscope 10. It should be noted that the band member 360 may be mounted on the operating part 14 of the endoscope 10 after the first connection tube 302 and the second connection tube 304 are held by the tube holding part 362.

According to the eighth embodiment, the first connection tube 302 and the second connection tube 304 are held on the operating part 14 of the endoscope 10 via the band member 360, so that these tubes do not interfere with the operation of the endoscope 10, and the technique is facilitated.

In the eighth embodiment, as an example, the case has been described in which the tube holding part 362 of the band member 360 holds two tubes (first connection tube 302 and second connection tube 304), but the present invention is not limited to this, and the tube holding part 362 may hold only one tube. In this case, a plurality of band members 360 different for each tube may be provided. The tube holding part 362 of the band member 360 may hold three or more tubes.

In the eighth embodiment, as an example, the configuration has been described in which the other end part of the tube holding part 362 is attachable to and detachable from the outer side surface of the band member 360, but the present invention is not limited to this, and, for example, the tube holding part 362 may have a loop shape, and both ends thereof may be fixed to the outer side surface of the band member 360.

In addition, in the eighth embodiment, as an example, the configuration has been described in which the first connection tube 302 and the second connection tube 304 are held by the tube holding part 362 of the band member 360, but any tube other than the first connection tube 302 and the second connection tube 304 may be used as long as the tube is used for the air supply/suction with respect to the first balloon 120 or the second balloon 220. For example, an extension tube that connects the first connection tube 302 and the first air supply port 132 (or the second connection tube 304 and the second air supply port 232) may be used.

In addition, in the eighth embodiment, as an example, the case has been described in which the holding member is composed of the band member 360, but the present invention is not limited to this, and the holding member may be composed of, for example, a string-like member as long as the band member 360 can be fixed to the operating part 14 of the endoscope 10.

### Ninth Embodiment

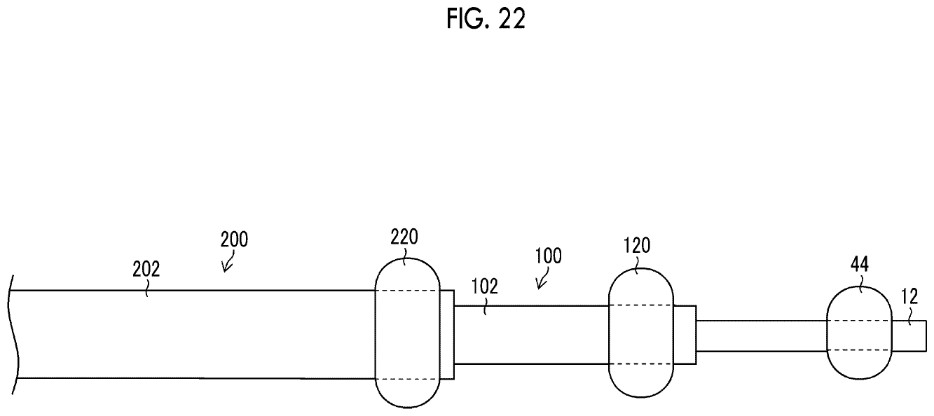

FIG. 22 is an explanatory diagram showing a ninth embodiment. The ninth embodiment is the same as the first embodiment except that the configuration of the distal end part of the endoscope insertion part 12 is different. Hereinafter, the difference from the first embodiment will be described.

As shown in FIG. 22, a third balloon 44 formed of various elastic bodies is provided on the outer peripheral surface of the distal end side of the endoscope insertion part 12. Since the third balloon 44 has essentially the same configuration as the first balloon 120 and the second balloon 220, the detailed description thereof will be omitted here.

Although not shown, the balloon control device 300 is further provided with, in addition to the control system of the first balloon 120 and the control system of the second balloon 220, a pressurization pump, a decompression pump, a solenoid valve, and a pressure sensor, which are a control system of the third balloon 44, and these pumps and the solenoid valve are controlled by the controller 322 (see FIG. 5). As a result, the third balloon 44 is expanded and contracted by the air supply/suction via the balloon control device 300.

The insertion method of the endoscope insertion part 12 according to the ninth embodiment is essentially the same as the insertion method according to the first embodiment described above, but has the following different points.

In the first embodiment, in a case in which the forward/backward movement operation of the inner tube 100 and the outer tube 200 is repeatedly performed, a so-called cantilever beam with a free distal end side is obtained in which the distal end part of the endoscope insertion part 12 is not fixed to the intestinal tract.

Meanwhile, in the ninth embodiment, since the third balloon 44 is mounted on the distal end part of the endoscope insertion part 12, the distal end part of the endoscope insertion part 12 can be fixed to the intestinal tract by expanding the third balloon 44 to be locked to the inner wall surface of the intestinal tract in a case in which the forward/backward movement operation of the inner tube 100 and the outer tube 200 is repeatedly performed. Therefore, even in a case in which the protruding region of the endoscope insertion part 12 that protrudes from the inner tube 100 is long, the forward/backward movement operation of the inner tube 100 and the outer tube 200 can be performed in a stable state by fixing the distal end part of the endoscope insertion part 12 to the intestinal tract.

### Other

The hardware structure that executes various types of control of the balloon control device 300 according to each of the above-described embodiments is various processors as shown below. The various processors include a central processing unit (CPU), which is a general-purpose processor that executes software (program) and functions as the various controllers, a programmable logic device (PLD), which is a processor of which a circuit configuration can be changed after manufacture, such as a field-programmable gate array (FPGA), and a dedicated electric circuit, which is a processor having a circuit configuration that is designed for exclusive use in order to execute specific processing, such as an application-specific integrated circuit (ASIC).

One processing unit may be configured by one of these various processors, or may be configured by two or more processors of the same type or different types (for example, a plurality of FPGAs, or a combination of a CPU and an FPGA). A plurality of controllers may be configured by one processor. As an example of configuring the plurality of controllers with one processor, first, as represented by a computer such as a client or a server, there is a form of configuring one processor with a combination of one or more CPUs and software and causing the processor to function as the plurality of controllers. Second, there is a form in which a processor, which implements the functions of the entire system including the plurality of controllers with one integrated circuit (IC) chip, is used, as represented by a system on a chip (SoC) or the like. As described above, various controllers are configured by one or more of the various processors described above, as the hardware structure.

In addition, the above-described embodiments may be combined with each other in any combination.

The embodiments of the present invention have been described above, but the present invention is not limited to the above-described examples, and various improvements or modifications may be made without departing from the scope of the present invention.

### EXPLANATION OF REFERENCES

1: endoscope system
10: endoscope
12: endoscope insertion part
14: operating part
16: universal cable
20: distal end part
22: bendable part
24: soft part
30: air/water supply button
32: suction button
34: angle knob
36: treatment tool inlet port
38: distal end surface
40: indicator
42: stopper part for endoscope insertion part
42a: stopper surface for endoscope insertion part
44: third balloon
50: overtube
52: endoscope insertion path
100: inner tube
102: inner tube body
104: inner tube gripping part
104a: small-diameter part
106: first insertion path
108: base end opening
110: distal end opening
120: first balloon
120a: attachment part
120b: attachment part
120c: bulging part
121: thread
122: first air supply/discharge pipe line
124: first liquid pipe line
124a: branch pipe line
124b: branch pipe line
126: first air supply/suction port
128: first liquid supply port
130: first balloon air supply pipe
132: first air supply port
134: first liquid supply pipe
136: first injection port
140: inner supply port
142: outer supply port
150: first sensor
152: indicator
154: first finger hook part
160: common pipe line
160a: branch part
162: air supply pipe line
164: suction pipe line
166: air supply source pipe line
168: suction source pipe line
170: valve housing part
172: communication space
174: pipe line switching valve
176: valve body part
176a: shaft part
176b: head part
176c: flange part
178: sealing member
180: spring member
182: first communication path
184: second communication path
186: stopper part for inner tube
186a: stopper surface for inner tube
200: outer tube
202: outer tube body
204: outer tube gripping part
204a: small-diameter part
206: second insertion path
208: base end opening
210: distal end opening
220: second balloon
220a: attachment part
220b: attachment part
220c: bulging part
221: thread
222: second air supply/discharge pipe line
224: second liquid pipe line
226: second air supply/suction port
228: second liquid supply port
230: second balloon air supply pipe
232: second air supply port
234: second liquid supply pipe
236: second injection port
250: second sensor
254A: second finger hook part
254B: second finger hook part
300: balloon control device
302: first connection tube
304: second connection tube
306: remote controller
310: pressurization pump
312: decompression pump
314: solenoid valve
316: pressurization pump
318: decompression pump
320: solenoid valve
322: controller
324: tube
326: tube
328: tube
330: tube
332: tube
334: tube
336: pressure sensor
338: pressure sensor
350: intestinal tract
360: band member
362: tube holding part
364: hook surface
366: loop surface
K: central axis

What is claimed is:

1. An overtube that is long and flexible, the overtube comprising:
an inner tube having a first insertion path into which an endoscope insertion part is insertable;
an outer tube having a second insertion path into which the inner tube is insertable;
a first balloon provided at a distal end part of the inner tube; and
a second balloon provided at a distal end part of the outer tube,
wherein an effective length of the outer tube is 1200 mm or longer and 1700 mm or shorter,
an effective length of the inner tube is 1400 mm or longer and 2000 mm or shorter,
in a case in which the endoscope insertion part is inserted into the first insertion path and the inner tube is inserted into the second insertion path, a total maximum protrusion length, which is a sum of a first maximum protrusion length of the endoscope insertion part from a distal end of the inner tube and a second maximum protrusion length of the inner tube from a distal end of the outer tube, is 300 mm or longer and 800 mm or shorter,
the first maximum protrusion length is 100 mm or longer and 550 mm or shorter,
the second maximum protrusion length is 200 mm or longer and 700 mm or shorter, and
a maximum protrusion length ratio, which is a ratio of the first maximum protrusion length to the second maximum protrusion length, is 0.15 or more and 1.0 or less.

2. The overtube according to claim 1,
wherein an axial length of at least one balloon of the first balloon or the second balloon is 50 mm or longer.

3. The overtube according to claim 2,
wherein the balloon has a bulging part that is expandable and contractable, and
an axial length of the bulging part is 30 mm or longer.

4. The overtube according to claim 1, further comprising:
an inner tube gripping part provided at a base end part of the inner tube; and
an outer tube gripping part provided at a base end part of the outer tube,
wherein the inner tube gripping part and the outer tube gripping part each have an axial length of 15 mm or longer and 200 mm or shorter.

5. The overtube according to claim 1,
wherein the inner tube has a stopper part for an inner tube in an inner tube protruding region that protrudes from the distal end of the outer tube, and
the stopper part for an inner tube has a stopper surface for an inner tube that comes into contact with the distal end of the outer tube to restrict a movement of the inner tube toward an axial base end side with respect to the outer tube.

6. The overtube according to claim 5,
wherein the stopper part for an inner tube is a large-diameter part having an outer diameter larger than an inner diameter of the second insertion path.

7. The overtube according to claim 5,
wherein the stopper part for an inner tube is provided at a position that is on a base end side with respect to the first balloon and that is within 100 mm from the distal end of the inner tube toward the base end side.

8. The overtube according to claim 5,
wherein the stopper part for an inner tube is attachable to and detachable from the inner tube.

9. The overtube according to claim 1,
wherein the inner tube has a lubricant supply part that supplies a lubricant, and
the lubricant supply part has an inner supply port that is open to an inner peripheral surface of the inner tube and an outer supply port that is open to an outer peripheral surface of the inner tube.

10. The overtube according to claim 9,
wherein the inner tube has an injection port for the lubricant, and
the injection port is in communication with the inner supply port and the outer supply port.

11. The overtube according to claim 1,
wherein the inner tube has an indicator indicating a relative axial position to the outer tube.

12. The overtube according to claim 1, further comprising:
an inner tube gripping part provided at a base end part of the inner tube; and
an outer tube gripping part provided at a base end part of the outer tube.

13. The overtube according to claim 12,
wherein the inner tube gripping part has a first finger hook part on which an operator is able to hook a finger, and
the outer tube gripping part has a second finger hook part on which the operator is able to hook a finger other than the finger.

14. The overtube according to claim 12,
wherein the inner tube gripping part is attachable to and detachable from the inner tube.

15. The overtube according to claim 1, further comprising:
a relative movement detection unit that detects a relative axial movement between the outer tube and the inner tube.

16. The overtube according to claim 15,
wherein the relative movement detection unit includes a proximity sensor or a position sensor.

17. The overtube according to claim 1, further comprising:
a movement direction detection unit that detects a movement direction of an axial movement of the inner tube.

18. The overtube according to claim 17,
wherein the movement direction detection unit includes an azimuth sensor or an angular velocity sensor.

19. The overtube according to claim 1,
wherein at least one tube of the inner tube or the outer tube includes
a common pipe line having one end side in communication with a balloon provided in the tube out of the first balloon and the second balloon,
an air supply pipe line and a suction pipe line that are branched at and connected to the other end side of the common pipe line,
an air supply source pipe line in communication with an air supply source,
a suction source pipe line in communication with a suction source,
a pipe line switching part that selectively switches between an air supply state in which the air supply pipe line and the air supply source pipe line are in communication with each other and the suction pipe line and the suction source pipe line are blocked from each other, and a suction state in which the air supply pipe line and the air supply source pipe line are blocked from each other and the suction pipe line and the suction source pipe line are in communication with each other,
a first communication path communicating between the air supply source pipe line and a space on an outer peripheral surface side or an inner peripheral surface side of the tube, and
a second communication path communicating between the suction source pipe line and the space on the outer peripheral surface side or the inner peripheral surface side of the tube.

20. An endoscope system comprising:
the overtube according to claim 1; and
a balloon control device that controls air supply and suction with respect to the first balloon and the second balloon,
wherein the overtube includes
a relative movement detection unit that detects a relative axial movement between the outer tube and the inner tube, and
a movement direction detection unit that detects a movement direction of an axial movement of the inner tube,
the balloon control device includes at least one processor, and
the processor is configured to
start the suction with respect to the second balloon in a case in which the relative movement detection unit detects that the distal end of the outer tube and the distal end of the inner tube are moved in a spaced direction,
start the air supply with respect to the second balloon in a case in which the relative movement detection unit detects that the distal end of the outer tube and the distal end of the inner tube are moved in an approaching direction,
start the air supply with respect to the first balloon in a case in which the movement direction detection unit detects that the inner tube is moved toward an axial distal end side, and
start the suction with respect to the first balloon in a case in which the movement direction detection unit detects that the inner tube is moved toward an axial base end side.

21. The endoscope system according to claim 20, further comprising:
a remote controller connected to the balloon control device,
wherein notification of a detection result of each of the relative movement detection unit and the movement direction detection unit is sent to the balloon control device via the remote controller.

22. An endoscope system comprising:
the overtube according to claim 1; and
an endoscope having the endoscope insertion part,
wherein the endoscope insertion part has a stopper part for an endoscope insertion part in an endoscope insertion part protruding region that protrudes from the distal end of the inner tube, and
the stopper part for an endoscope insertion part has a stopper surface for an endoscope insertion part that comes into contact with the distal end of the inner tube to restrict a movement of the endoscope insertion part toward an axial base end side with respect to the inner tube.

23. The endoscope system according to claim 22,
wherein the stopper part for an endoscope insertion part is a large-diameter part having an outer diameter larger than an inner diameter of the first insertion path.

24. The endoscope system according to claim 22,
wherein the stopper part for an endoscope insertion part is attachable to and detachable from the endoscope insertion part.

25. An endoscope system comprising:
the overtube according to claim 1;
an endoscope having the endoscope insertion part and an operating part installed consecutively on a base end side of the endoscope insertion part; and
a holding member provided to be attachable to and detachable from the operating part and holding at least one tube of a first tube for performing air supply and suction with respect to the first balloon and a second tube for performing air supply and suction with respect to the second balloon.

26. An endoscope system comprising:
the overtube according to claim 1; and
an endoscope having the endoscope insertion part,
wherein an effective length of the endoscope insertion part is 2000 mm.

27. An endoscope system comprising:
the overtube according to claim 1; and
an endoscope having the endoscope insertion part,
wherein a third balloon is provided at a distal end part of the endoscope insertion part.

28. An operation method of an overtube for operating the overtube according to claim 1, in a state in which the overtube and the endoscope insertion part are inserted into an intestinal tract, the inner tube is inserted into the second insertion path such that a part of the inner tube is exposed from the distal end of the outer tube, and the endoscope insertion part is inserted into the first insertion path such that a part of the endoscope insertion part is exposed from the distal end of the inner tube, the operation method comprising:
an inner tube pushing-in step of pushing in the inner tube such that the first balloon and the second balloon are spaced from each other in a range in which the distal end of the inner tube is located on a base end side with respect to the distal end of the endoscope insertion part in a state in which the first balloon is contracted;
a first balloon expansion step of expanding the first balloon; an outer tube pushing-in step of pushing in the outer tube such that the second balloon approaches the first balloon in a state in which the second balloon is contracted;
a second balloon expansion step of expanding the second balloon; and
a retraction step of retracting the inner tube and the outer tube to pull in the intestinal tract in a state in which the first balloon and the second balloon are expanded and are close to each other.

29. The operation method of an overtube according to claim 28,
wherein the second balloon expansion step and the retraction step are performed in parallel.

30. The operation method of an overtube according to claim 28,
wherein the inner tube and the outer tube are operated by a second operator different from a first operator who operates the endoscope insertion part.

31. The operation method of an overtube according to claim 28,
wherein the inner tube has a stopper part for an inner tube in an inner tube protruding region that protrudes from the distal end of the outer tube,
the stopper part for an inner tube has a stopper surface for an inner tube that comes into contact with the distal end of the outer tube to restrict a movement of the inner tube toward an axial base end side with respect to the outer tube, and
the inner tube and the outer tube are retracted together by retracting the inner tube in a state in which the stopper surface for an inner tube is in contact with the distal end of the outer tube in the retraction step.

32. The operation method of an overtube according to claim 31,
wherein the inner tube is operated by a second operator different from a first operator who operates the endoscope insertion part.

33. An insertion method of an endoscope insertion part comprising:
the operation method of an overtube according to claim 28; and
an endoscope insertion part pushing-in step of pushing in the endoscope insertion part such that the distal end of the endoscope insertion part is spaced from the distal end of the inner tube in a state in which a part of the endoscope insertion part is exposed from the distal end of the inner tube.

34. The insertion method of an endoscope insertion part according to claim 33, further comprising
a repeat step of performing, in a case in which an operation including the inner tube pushing-in step, the first balloon expansion step, the outer tube pushing-in step, the second balloon expansion step, and the retraction step is defined as an overtube operation, the overtube operation at least one time,
wherein the endoscope insertion part pushing-in step is performed independently of the repeat step.

35. The insertion method of an endoscope insertion part according to claim 34,
wherein the endoscope insertion part pushing-in step is performed in a case in which the pulled-in intestinal tract is accumulated on a base end side of the second balloon due to the repeat step.

36. The insertion method of an endoscope insertion part according to claim 34,
wherein the endoscope insertion part pushing-in step is performed in a case in which the pulled-in intestinal tract is accumulated on the base end side of the second balloon due to the repeat step, and then the repeat step is further performed.
